(19) United States
(12) Patent Application Publication
(10) Pub. No.: US 2002/0099073 A1
(43) Pub. Date: Jul. 25, 2002

(54) Modulators of protein tyrosine phosphatases (PTPases)

(76) Inventors: Henrik Sune Andersen, Lyngby (DK); Thomas Kruse Hansen, Herlev (DK); Jesper Lau, Farum (DK); Niels Peter Hundahl Moller, Kobenhavn O (DK); Ole Hvilsted Olsen, Bronshoj (DK); Frank Urban Axe, Escondido, CA (US); Farid Bakir, San Diego, CA (US); Yu Ge, San Diego, CA (US); Daniel Dale Holsworth, San Diego, CA (US); Luke Milburn Judge, Seattle, WA (US); Michael James Newman, San Diego, CA (US); Roy Teruyuki Uyeda, San Diego, CA (US); Barry Zvi Shapira, Acton, CA (US)

Correspondence Address: Reza Green, Esq., Novo Nordisk of North America, Inc., Suite 6400, 405 Lexington Avenue, New York, NY, 10174-6401, US

(21) Appl. No.: 09/901,284

(22) Filed: Jul. 9, 2001

Related U.S. Application Data
(60) Provisional application No. 60/235,615, filed on Sep. 27, 2000.

(30) Foreign Application Priority Data
Jul. 7, 2000 (DK) PA 2000 01066

(51) Int. Cl. C07D 498/02; A61K 31/4743

(52) U.S. Cl. 514/301; 546/114

(57) ABSTRACT

The present invention provides novel thienopyridines, novel compositions, methods of their use, and methods of their manufacture, where such compounds of Formula 1 are pharmacologically useful inhibitors of Protein Tyrosine Phosphatases (PTPase's) including PTP1B, T cell PTP (TC-PTP),

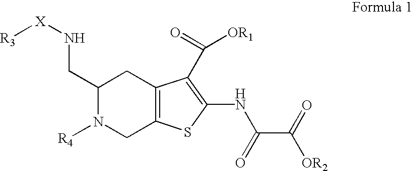

wherein X, R₁, R₂, R₃, and R₄ are defined more fully in the description. The compounds are useful in the treatment of type 1 diabetes, type 2 diabetes, impaired glucose tolerance, insulin resistance, obesity, and other diseases.

### CROSS REFERENCE TO RELATED APPLICATIONS

This application claims priority under 35 U.S.C. 119 of Danish application no. PA 2000 01066 filed on Jul. 7, 2000, and U.S. provisional application no. 60/235,615 filed on Sep. 27, 2000, the contents of which are fully incorporated herein by reference.

### FIELD OF THE INVENTION

The present invention relates to novel thienopyridines, to methods for their preparation, to compositions comprising the compounds, to the use of these compounds as medicaments and their use in therapy, where such compounds of Formula 1 are pharmacologically useful inhibitors or modulators of Protein Tyrosine Phosphatases (PTPases) including PTP1B and T cell PTP (TC-PTP),

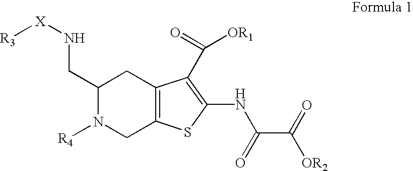

wherein X, R₁, R₂, R₃, and R₄ are defined more fully below.

It has been found that PTPases play a major role in the modulation and regulation of fundamental cellular signaling mechanisms involved in metabolism, growth, proliferation and differentiation (Fischer et al, Science 253:401-6 (1991); Tonks and Neel, Cell 87: 365-368 (1996); Neel and Tonks, Current Opinion in Cell Biology 9: 193-204 (1997); Hunter, Phil. Trans. R. Soc. Lond. B 353: 583-605 (1998); Zhang, Critical Reviews in Biochemistry and Molecular Biology 33:1-52 (1998)). There is increasing evidence which suggests that inhibition of these PTPases may help treat or manage certain types of diseases such as type 1 and type 2 diabetes, obesity, autoimmune diseases, acute and chronic inflammation, osteoporosis and various forms of cancer. In addition, certain infectious diseases may also be treated or managed by administration PTPase inhibitors (Clemens et al., Molecular Microbiology 5: 2617-2620 (1991)).

### BACKGROUND OF THE INVENTION

Protein phosphorylation is now well recognized as an important mechanism utilized by cells to transduce and regulate signals during different stages of cellular function (Hunter, vide supra; Chan et al., Annu. Rev. Immunol. 12: 555-592 (1994); Zhang, Curr. Top. Cell. Reg. 35: 21-68 (1997); Matozaki and Kasuga, Cell. Signal. 8: 113-19 (1996); Fischer et al, vide supra). The level of tyrosine phosphorylation is balanced by the opposing action of protein tyrosine kinases and protein tyrosine phosphatases (PTPases). There are at least two major classes of phosphatases: (1) those that dephosphorylate proteins (or peptides) that contain a phosphate group(s) on a serine or threonine moiety (termed Ser/Thr phosphatases) and (2) those that remove a phosphate group(s) from the amino acid tyrosine (termed protein tyrosine phosphatases or PTPases or PTPs). The PTPases are a family of enzymes that can be classified into two groups: a) intracellular or nontransmembrane PTPases and b) receptor-type or transmembrane PTPases. In addition, dual-specificity phosphatases and low molecular weight phosphatases can also dephosphorylate phosphotyrosyl proteins (WO97/39748, WO97/40017, WO99/15529, WO97/08934, WO98/27065, WO99/46236, WO99/46244, WO99/46267, WO99/46268, WO99/46237).

It has been found that PTPases play a major role in the above modulation and regulation of fundamental cellular signaling mechanisms involved in metabolism, growth, proliferation and differentiation (Fischer et al, Science 253:401-6 (1991); Tonks and Neel, Cell 87: 365-368 (1996); Neel and Tonks, Current Opinion in Cell Biology 9: 193-204 (1997); Hunter, Phil. Trans. R. Soc. Lond. B 353: 583-605 (1998); Zhang, Critical Reviews in Biochemistry and Molecular Biology 33:1-52 (1998)). Reports from many laboratories have shown that PTPases can act both as positive and negative regulators of signal transduction processes. PTPases have been implicated in a variety of human diseases, including type 1 and type 2 diabetes, obesity, autoimmune diseases, acute and chronic inflammation, osteoporosis, proliferative disorders including various forms of cancer, growth disorders, and defective platelet aggregation (WO97/39748, WO97/40017, WO99/15529, WO97/08934, WO98/27065, WO99/46236, WO99/46244, WO99/46267, WO99/46268, WO99/46237). Accordingly, there is increasing evidence which suggests that inhibition of these PTPases may help treat or manage said diseases (Hunter, vide supra; Neel and Tonks, vide supra; Frangione et al., EMBO J. 12: 4843-4856; Zhang, Curr. Top. Cell. Reg. 35: 21-68 (1997); Zhang, vide supra; Evans and Jallan, Exp. Opinion. Invest. Drugs 8: 139-160 (1999); Burke and Zhang, Biopolymers (Peptide Science) 47: 225-241 (1998); Elchebly et al., Science 283: 1544-1548 (1999); Wrobel et al., J. Med. Chem. 42: 3199-3202 (1999)). In addition, certain infectious diseases may also be treated or managed by administration PTPase inhibitors (Clemens et al., Molecular Microbiology 5: 2617-2620 (1991)).

Both selective PTPase inhibitors and inhibitors that bind to several PTPases (non-selective inhibitors) can be used therapeutically to partially or completely restore PTPase-mediated perturbed signal transduction processes and thus for management, treatment or prevention of the above diseases.

WO 99/46267 discloses compounds, which are pharmacologically useful inhibitors of PTPases. However, the present invention, which represents a novel selection under WO 99/46267, discloses a class of compounds, which surprisingly are more potent against protein tyrosine phosphatases (e.g. PTPI B) than those disclosed in WO 99/46267.

### DESCRIPTION OF THE INVENTION

The present invention relates to compounds of the Formula 1 wherein X, R₁, R₂, R₃, and R₄ are defined below;

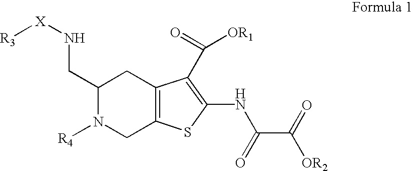

wherein

X is —C(O)— or —S(O)₂—;

R₁ and R₂ are independently hydrogen, C₁-C₆alkyl, aryl-R₅—, R₆—C(O)—O—R₇— or aryl-R₈—C(O)—O—R₉— wherein aryl is phenyl, naphthyl or thiophenyl, which aryl group is optionally substituted with halogen, nitro, trihalomethyl, C₁-C₆alkyl or C₁-C₆alkyloxy;

R₃ is hydrogen, C₁-C₆alkyl, H₂N—R₃₇—, C₂-C₆alkenyl, C₂-C₆alkynyl, aryl, aryl-R₁₀—, aryl-N(R₃₅)—, aryl-R₁₁—N(R₃₆)—, C₁-C₆alkyloxy or aryl-R₁₃—O— wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, which aryl group is optionally substituted with halogen, nitro, cyano, hydroxy, trihalomethyl, C₁-C₆alkyl, R₃₈-aryl-, aryl, aryl-R₁₄—, C₁-C₆alkyloxy, aryloxy, aryl-R₁₅—O—, aryl-N(R₁₆)—, R₁₈—C(O)—N(R₁₉)—, aryl-C(O)—N(R₂₁)— or aryl-R₂₃—C(O)—N(R₂₄)— and wherein aryl is phenyl, naphthyl or thiophenyl;

R₄ is hydrogen, C₁-C₆alkyl, aryl-R₂₆—, R₂₇—O—C(O)—, aryl-R₂₈—O—C(O)—, R₂₉—C(O)—O—R₃₀—O—C(O)— or aryl-R₃₁—C(O)—O—R₃₂—O—C(O)— wherein aryl is phenyl, naphthyl or thiophenyl, which aryl group is optionally substituted with halogen, nitro, cyano, trihalomethyl, aryl, aryl-R₃₃—, C₁-C₆alkyloxy or aryl-R₃₄—O— and wherein aryl group is phenyl, naphthyl or thiophenyl;

and wherein R₅, R₇, R₈, R₉, R₁₀, R₁₁, R₁₃, R₁₄, R₁₅, R₂₃, R₂₆, R₂₈, R₃₀, R₃₁, R₃₂, R₃₃, R₃₄, and R₃₇ independently are C₁₋₆-alkylene, wherein R₆, R₁₂, R₁₇, R₁₈, R₂₀, R₂₂, R₂₅, R₂₇ and R₂₉ independently are C₁₋₆alkyl and wherein R₁₆, R₁₉, R₂₁, R₂₄, R₃₅, R₃₆ and R₃₈ independently are hydrogen or C₁₋₆alkyl;

or a salt thereof with a pharmaceutically acceptable acid or base, or any optical isomer or mixture of optical isomers, including a racemic mixture, or any tautomeric form.

The compounds of the invention can be further modified to act as prodrugs.

A preferred prodrug is acetoxymethyl esters or acetoxymethyl carbamates of the compounds of the present invention. As a general procedure preparation of an acetoxymethyl ester is given below (C. Schultz et al, The Journal of Biological Chemistry, 1993, 268, 6316-6322.):

A carboxylic acid (1 equivalent) is suspended in dry acetonitrile (2 ml per 0.1 mmol). Diisopropyl amine (3.0 equivalents) is added followed by bromomethyl acetate (1.5 equivalents). The mixture is stirred under nitrogen overnight at room temperature. Acetonitrile is removed under reduced pressure to yield an oil which is diluted in ethyl acetate and washed with water (3×). The organic layer is dried over anhydrous magnesium sulfate, filtred and the solvent removal under reduced pressure affording a crude oil. The product is purified by column chromatography on silica gel, using an appropriate solvent system known to those skilled in the art.

Definitions

As used herein, the term “attached” or “—” signifies a stable covalent bond, certain preferred points of attachment points being apparent to those skilled in the art.

The terms “halogen” and “halo” includes fluorine, chlorine, bromine, and iodine.

The term “alkyl” includes C₁-C₆ straight chain saturated, C₁-C₆ branched chain saturated and C₃-C₆ cyclic saturated hydrocarbon groups. For example, this definition shall include but is not limited to methyl (Me), ethyl (Et), propyl (Pr), butyl (Bu), pentyl, hexyl, isopropyl (i-Pr), isobutyl (i-Bu), tert-butyl (t-Bu), sec-butyl (s-Bu), cyclopropyl, cyclobutyl, cyclopentyl, cyclohexyl and the like.

The term “alkenyl” includes C₂-C₆ unsaturated aliphatic hydrocarbon groups and C₂-C₆ branched unsaturated aliphatic hydrocarbon groups having the specified number of carbon atoms and at lest one double bond. For example, this definition shall include but is not limited to ethenyl, propenyl, butenyl, pentenyl, hexenyl, isopentenyl, neopentenyl, cyclobutenyl, cyclopentenyl, cyclohexenyl, and the like.

The term “alkynyl” includes C₂-C₆ straight chain unsaturated aliphatic, C₂-C₆ branched unsaturated and cyclic C₆ unsaturated aliphatic hydrocarbon groups having the specified number of carbon atoms and at lest one triple bond. For example, this definition shall include but is not limited to acetynyl, propynyl, butynyl, pentynyl, hexynyl, cyclohexynyl and the like.

The term “alkyloxy” (e.g. methoxy, ethoxy, propyloxy, allyloxy, cyclohexyloxy) represents an “alkyl” group as defined above having the indicated number of carbon atoms attached through an oxygen bridge.

The term “aryloxy” (e.g. phenoxy, naphthyloxy and the like) represents an aryl group as defined below attached through an oxygen bridge.

The term “aryl” represents an unsubstituted, mono-, di- or trisubstituted monocyclic, polycyclic, biaryl or heterocyclic aromatic group(s) covalently attached at any ring position capable of forming a stable covalent bond, certain preferred points of attachment being apparent to those skilled in the art (e.g., 3-indolyl, 4(5)-imidazolyl).

The definition of aryl includes phenyl, biphenyl, indenyl, naphthyl (1-naphthyl, 2-naphthyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), thiophenyl (2-thiophenyl, 3-thiophenyl, 4-thiophenyl, 5-thiophenyl), pyridyl (2-pyridyl, 3-pyridyl, 4-pyridyl, 5-pyridyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), indolyl (1-indolyl, 2-indolyl, 3-indolyl, 4-indolyl, 5-indolyl, 6-indolyl, 7-indolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl).

It is a well known problem in drug discovery that compounds, such as enzyme inhibitors, may be very potent and selective in biochemical assays, yet be inactive in vivo. This lack of so-called bioavailability may be ascribed to a number of different factors such as lack of or poor absorption in the gut, first pass metabolism in the liver, poor uptake in cells. Although the factors determining bioavailability are not completely understood, there are many examples in the scientific literature—well known to those skilled in the art—of how to modify compounds, which are potent and selective in biochemical assays but show low or no activity in vivo, into drugs that are biologically active. By the term ‘original compound’ is understood a compound of Formula 1 wherein R₁ and R₂ are both hydrogen. It is within the scope of the invention to modify the original compounds of the invention by attaching chemical groups that will improve the bioavailability of said compounds in such a way that the uptake in cells or mammals is facilitated. Examples of said modifications, which are not intended in any way to limit the scope of the invention, include changing of one or more of the carboxy groups at the R₁ and R₂ position to esters (for instance methyl esters, ethyl esters, acetoxymethyl esters or other acyloxymethyl esters). Original compounds of the invention modified by attaching chemical groups are termed ‘modified compounds’. Other examples of modified compounds, which are not intended in any way to limit the scope of the invention, are compounds that have been cyclized at specific positions—so called ‘cyclic compounds’—which upon uptake in cells or mammals become hydrolyzed at the same specific position(s) in the molecule to yield the compounds of the invention, the original compounds, which are then said to be ‘non-cyclic’. For the avoidance of doubt, it is understood that the latter original compounds in most cases will contain other cyclic or heterocyclic structures that will not be hydrolyzed after uptake in cells or mammals. Generally, said modified compounds may not show behavior in biochemical assays similar to that of the original compound, i.e. the corresponding compounds of the invention without the attached chemical groups or said modifications. Said modified compounds may even be inactive in biochemical assays. However, after uptake in cells or mammals these attached chemical groups of the modified compounds may in turn be removed spontaneously or by endogenous enzymes or enzyme systems to yield compounds of the invention, original compounds. ‘Uptake’ is defined as any process that will lead to a substantial concentration of the compound inside cells or in mammals. After uptake in cells or mammals and after removal of said attached chemical group or hydrolysis of said cyclic compound, the compounds may have the same structure as the original compounds and thereby regain their activity and hence become active in cells and/or in vivo after uptake. Thus, the term ‘a functional group which can be converted to hydrogen in vivo’ is intended to include any group which upon administering the present compounds to the subjects in need thereof can be converted to hydrogen e.g. enzymatically or by the acidic environment in the stomach.

The compounds of the present invention have asymmetric centres and may occur as racemates, racemic mixtures, and as individual enantiomers or diastereoisomers, with all isomeric forms being included in the present invention as well as mixtures thereof.

Pharmaceutically acceptable salts of the compounds of Formula 1, where a basic or acidic group is present in the structure, are also included within the scope of this invention. When an acidic substituent is present, such as —COOH, 5-tetrazolyl or —P(O)(OH)₂, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethane sulfonate, picrate and the like, and include acids related to the pharmaceutically acceptable salts listed in Journal of Pharmaceutical Science, 66, 2 (1977) and incorporated herein by reference, can be used as the dosage form.

Also, in the case of the —COOH or —P(O)(OH)₂ being present, pharmaceutically acceptable esters can be employed, e.g., methyl, tert-butyl, pivaloyloxymethyl, and the like, and those esters known in the art for modifying solubility or hydrolysis characteristics for use as sustained release or prodrug formulations.

In addition, some of the compounds of the present invention may form solvates with water or common organic solvents. Such solvates are encompassed within the scope of the invention.

The term “therapeutically effective amount” shall mean that amount of drug or pharmaceutical agent that will elicit the biological or medical response of a tissue, system, animal, or human that is being sought by a researcher, veterinarian, medical doctor or other.

### PREFERRED EMBODIMENTS OF THE INVENTION

In a preferred embodiment, the present invention is concerned with compounds of Formula 1

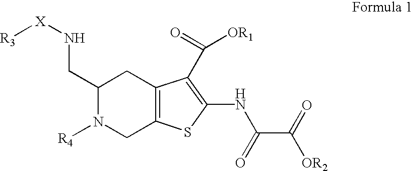

wherein

X is —C(O)—;

R₁ and R₂ are independently hydrogen, C₁-C₆alkyl, aryl-R₅—, R₆—C(O)—O—R₇— or aryl-R₈—C(O)—O—R₉— wherein aryl is phenyl, naphthyl, thiophenyl, which aryl group is optionally substituted with halogen, trihalomethyl, aryl, aryl-R₁₀—, C₁-C₆alkyloxy or aryl-R₁₁—O—;

R₃ is C₁-C₆alkyl, H₂N—R₃₅—, C₂-C₆alkenyl, C₂-C₆alkynyl, aryl-R₁₂—, aryl, aryl-R₁₃—, aryl-N(R₁₄)—, aryl-R₁₅—N(R₁₆)—, R₃₈-aryl-, C₁-C₆alkyloxy or aryl-R₁₇—O— wherein aryl is phenyl, biphenyl, naphthyl, 1,2,3-triazolyl, thiophenyl, pyridyl, quinolyl, isoquinolyl, or indolyl, which aryl group is optionally substituted with halogen, hydroxy, C₁-C₆alkyl, aryl, aryl-R₁₈—, C₁-C₆alkyloxy, aryloxy, aryl-R₁₉—O—;

R₄ is hydrogen, C₁-C₆alkyl, aryl-R₂₀—, R₂₁—C(O)—O—R₂₂—O—C(O)— or aryl-R₂₃—C(O)—O—R₂₄—O—C(O)— wherein aryl is phenyl or thiophenyl, which aryl group is optionally substituted with halogen, nitro, cyano, trihalomethyl, aryl, aryl-R₂₅—, C₁-C₆alkyloxy or aryl-R₂₆—O— and wherein aryl group is phenyl, naphthyl or thiophenyl; and

wherein R₅, R₇, R₈, R₉, R₁₀, R₁₁, R₁₃, R₁₅, R₁₇, R₁₈, R₁₉, R₂₀, R₂₂, R₂₃, R₂₄, R₂₅, R₂₆, and R₃₅ independently are C₁-C₆-alkylene, wherein R₁₂ is alkenylene, wherein R₆, R₂₁, and R₃₈ independently are C₁-C₆alkyl and wherein R₁₄ and R₁₆ independently are hydrogen or C₁₋₆alkyl;

or a salt thereof with a pharmaceutically acceptable acid or base, or any optical isomer or mixture of optical isomers, including a racemic mixture, or any tautomeric form.

More preferred compounds of the invention are compounds of Formula I

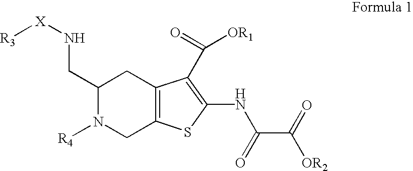

wherein

X is —C(O)—;

R₁ and R₂ are independently hydrogen or C₁-C₆alkyl;

R₃ is C₁-C₆alkyl, H₂N—R₁₈, aryl-R₅—, aryl, aryl-R₆—, aryl-N(R₇)—, aryl-R₈—O— or R₁₉-aryl- wherein aryl is phenyl, biphenyl, naphthyl, 1,2,3-triazolyl, thiophenyl, pyridyl, quinolyl, isoquinolyl, or indolyl, which aryl group is optionally substituted with halogen, hydroxy, C₁-C₆alkyl, aryl, aryl-R₉—, C₁-C₆alkyloxy, aryloxy, aryl-R₁₀—O—;

R₄ is hydrogen, aryl-R₁₁—, R₁₂—C(O)—O—R₁₃—O—C(O)— or aryl-R₁₄—C(O)—O—R₁₅—O—C(O)-wherein aryl is phenyl or thiophenyl, which aryl group is optionally substituted with halogen, nitro, cyano, trihalomethyl, aryl, aryl-R₁₆—, C₁-C₆alkyloxy or aryl-R₁₇—O— and wherein aryl group is phenyl, naphthyl or thiophenyl; and

wherein R₅, R₈, R₉, R₁₀, R₁₁, R₁₃, R₁₄, R₁₅, R₁₆, R₁₇ and R₁₈ independently are C₁-C₆-alkylene, wherein R₆ is alkenylene, wherein R₇ and R₁₂ independently are C₁-C₆alkyl and wherein R₇ and R₁₉ independently are hydrogen or C₁-C₆alkyl;

or a salt thereof with a pharmaceutically acceptable acid or base, or any optical isomer or mixture of optical isomers, including a racemic mixture, or any tautomeric form.

Even more preferred compounds of the invention are compounds of Formula

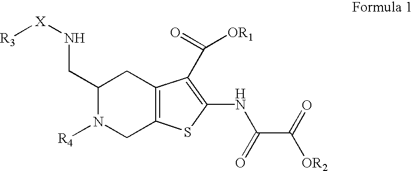

wherein

X is —C(O)—;

R₁, R₂ and R₄ are hydrogen;

R₃ is C₁-C₆alkyl, H₂N—R₁₂—, aryl, aryl-R₅—, aryl-N(R₆)—, aryl-R₇—O— or R₁₃-aryl-, wherein aryl is phenyl, biphenyl, naphthyl, 1,2,3-triazolyl, thiophenyl, pyridyl, quinolyl, isoquinolyl, or indolyl, which aryl group is optionally substituted with halogen, hydroxy, C₁-C₆alkyl, aryl, aryl-R₈—, C₁-C₆alkyloxy, aryloxy, aryl-R₉—O-wherein aryl is phenyl or thiophenyl, which aryl group is optionally substituted with halogen, hydroxy, nitro, cyano, trihalomethyl, aryl, aryl-R₁₀—, C₁-C₆alkyloxy aryloxy, or aryl-R₁₁—O— and wherein aryl group is phenyl, naphthyl or thiophenyl;

wherein R₅, R₇, R₈, R₉, R₁₀, R₁₁ and R₁₂ independently are C₁-C₆-alkylene and wherein R₆ and R₁₃ independently are hydrogen or C₁₆alkyl;

or a salt thereof with a pharmaceutically acceptable acid or base, or any optical isomer or mixture of optical isomers, including a racemic mixture, or any tautomeric form.

The following compounds are preferred:

5-(Benzoylamino-methyl)-2-(oxalyl-amino)-4,5,6,7-tetrahydro-thieno[2,3-c] pyridine-3-carboxylic acid;

5-(((6-Bromo-2-p-tolyl-quinoline-4-carbonyl)amino)methyl)-6-(4-methoxy-benzyl)-2-(oxalyl-amino)-4,5,6,7-tetrahydro-thieno[2,3-c]pyridine-3-carboxylic acid;

5-(((5-Benzyloxy-1H-indole-2-carbonyl)amino)methyl)-6-(4-methoxy-benzyl)-2-(oxalyl-amino)-4,5,6,7-tetrahydro-thieno[2,3-c]pyridine-3-carboxylic acid;

5-((4-Benzoylamino-benzoylamino)methyl)-6-(4-methoxy-benzyl)-2-(oxalyl-amino)-4,5,6,7-tetrahydro-thieno[2,3-c]pyridine-3-carboxylic acid;

5-((4-Benzoylamino-benzoylamino)methyl)-2-(oxalyl-amino)-4,5,6,7-tetrahydro-thieno[

2,3-c]pyridine-3-carboxylic acid;

6-(4-Methoxy-benzyl)-5-(((5-methyl-2-phenyl-2H-[1,2,3]triazole-4-carbonyl)amino)methyl)-2-(oxalyl-amino)-4,5,6,7-tetrahydro-thieno[2,3-c]pyridine-3-carboxylic acid;

5-((4-Ethoxy-2-hydroxy-benzoylamino)-methyl)-6-(4-methoxy-benzyl)-2-(oxalyl-amino)-4,5,6,7-tetrahydro-thieno[2,3-c]pyridine-3-carboxylic acid;

5-(((Biphenyl-4-carbonyl)-amino)-methyl)-6-(4-methoxy-benzyl)-2-(oxalyl-amino)-4,5,6,7-tetrahydro-thieno[2,3-c]pyridine-3-carboxylic acid;

5-(((Biphenyl-4-carbonyl)amino)methyl)-2-(oxalyl-amino)-4,5,6,7-tetrahydro-thieno[

2,3-c]pyridine-3-carboxylic acid;

5-((4-Ethoxy-2-hydroxy-benzoylamino)methyl)-2-(oxalyl-amino)-4,5,6,7-tetrahydro-thieno[2,3-c]pyridine-3-carboxylic acid;

5-(((5-Methyl-2-phenyl-2H-[1,2,3]triazole-4-carbonyl)amino)methyl)-2-(oxalyl-amino)-4,5,6,7-tetrahydro-thieno[2,3-c]pyridin-3-carboxylic acid;

5-((3-Biphenyl-4-yl-acryloylamino)methyl)-6-(4-methoxy-benzyl)-2-(oxalyl-amino)-4,5,6,7-tetrahydro-thieno[2,3-c]pyridin-3-carboxylic acid;

5-(((1H-Indole-2-carbonyl)amino)methyl)-6-(4-methoxy-benzyl)-2-(oxalyl-amino)-4,5,6,7-tetrahydro-thieno[2,3-c]pyridin-3-carboxylic acid;

5-(((1H-Indole-2-carbonyl)amino)methyl)-2-(oxalyl-amino)-4,5,6,7-tetrahydro-thieno[

2,3-c]pyridine-3-carboxylic acid;

5-((3-Biphenyl-4-yl-propionylamino)methyl)-2-(oxalyl-amino)-4,5,6,7-tetrahydro-thieno[

2,3-c]pyridine-3-carboxylic acid;

6-(4-Methoxy-benzyl)-5-(((naphthalene-1-carbonyl)-amino)-methyl)-2-(oxalyl-amino)-4,5,6,7-tetrahydro-thieno[2,3-c]pyridine-3-carboxylic acid;

5-(Benzyloxycarbonylamino-methyl)-2-(oxalyl-amino)-4,5,6,7-tetrahydro-thieno[

2,3-c]pyridine-3-carboxylic acid;

7-(benzyloxycarbonylamino-methyl)-2-(oxalyl-amino)-4,5,6,7-tetrahydro-thieno[

2,3-c]pyridine-3-carboxylic acid;

5-((2-Hydroxy-benzoylamino)-methyl)-2-(oxalyl-amino)-4,5,6,7-tetrahydro-thieno[

2,3-c]pyridine-3-carboxylic acid;

5-((3-Hydroxy-benzoylamino)-methyl)-2-(oxalyl-amino)-4,5,6,7-tetrahydro-thieno[

2,3-c] pyridine-3-carboxylic acid;

2-(Oxalyl-amino)-5-(S)-((3-phenoxy-benzoylamino)methyl)-4,5,6,7-tetrahydro-thieno[

2,3-c]pyridine-3-carboxylic acid;

5-((4-Benzoylamino-benzoylamino)methyl)-2-(oxalyl-amino)-4,5,6,7-tetrahydro-thieno[

2,3-c]pyridine-3-carboxylic acid;

5-(R)-((4-Benzoylamino-benzoylamino)methyl)-2-(oxalyl-amino)-4,5,6,7-tetrahydro-thieno[2,3-c]pyridine-3-carboxylic acid;

5-(S)-(((1-Hydroxy-naphthalene-2-carbonyl)amino)methyl)-2-(oxalyl-amino)-4,5,6,7-tetrahydro-thieno[2,3-c]pyridine-3-carboxylic acid;

5-(S)-(((3-Hydroxy-naphthalene-2-carbonyl)amino)methyl)-2-(oxalyl-amino)-4,5,6,7-tetrahydro-thieno[2,3-c]pyridine-3-carboxylic acid;

5-(S)-(((3-Hydroxy-7-methoxy-naphthalene-2-carbonyl)amino)methyl)-2-(oxalyl-amino)-4,5,6,7-tetrahydro-thieno[2,3-c]pyridine-3-carboxylic acid;

2-(Oxalyl-amino)-5-(S)-((3-phenoxy-benzoylamino)methyl)-4,5,6,7-tetrahydro-thieno[

2,3-c]pyridine-3,6-dicarboxylic acid 6-acetoxymethyl ester;

2-(Oxalyl-amino)-5-(S)-(3-(4-phenoxy-phenyl)ureidomethyl)-4,5,6,7-tetrahydro-thieno[

2,3-c]pyridin-3-carboxylic acid;

5-((4-Benzyl-benzoylamino)methyl)-2-(oxalyl-amino)-4,5,6,7-tetrahydro-thieno[

2,3-c]pyridine-3-carboxylic acid;

5-((5-Amino-5-methyl-hexanoylamino)methyl)-2-(oxalyl-amino)-4,5,6,7-tetrahydro-thieno[2,3-c]pyridine-3-carboxylic acid;

5-(S)-(((Biphenyl-4-carbonyl)amino)methyl)-6-methyl-2-(oxalyl-amino)-4,5,6,7-tetrahydro-thieno[2,3-c]pyridine-3-carboxylic acid;

5-(S)-((4-Benzyl-benzoylamino)methyl)-6-methyl-2-(oxalyl-amino)-4,5,6,7-tetrahydro-thieno[2,3-c]pyridine-3-carboxylic acid;

5-(S)-(((Naphthalene-2-carbonyl)amino)methyl)-2-(oxalyl-amino)-4,5,6,7-tetrahydro-thieno[2,3-c]-pyridine-3-carboxylic acid;

5-(S)-(((1H-Indole-2-carbonyl)amino)methyl)-2-(oxalyl-amino)-4,5,6,7-tetrahydro-thieno[

2,3-c]pyridine-3-carboxylic acid;

5-(S)-(((1H-Indole-2-carbonyl)amino)methyl)-6-methyl-2-(oxalyl-amino)-4,5,6,7-tetrahydro-thieno[2,3-c]pyridine-3-carboxylic acid;

6-Benzyl-5-(S)-(((naphthalene-2-carbonyl)amino)methyl)-2-(oxalyl-amino)-4,5,6,7-tetrahydro-thieno[2,3-c]pyridine-3-carboxylic acid;

6-Benzyl-5-(S)-(((1H-indole-2-carbonyl)amino)methyl)-2-(oxalyl-amino)-4,5,6,7-tetrahydro-thieno[2,3-c]pyridine-3-carboxylic acid;

or a pharmaceutically acceptable salt thereof.

In another embodiment the invention relates to a compound of Formula 1

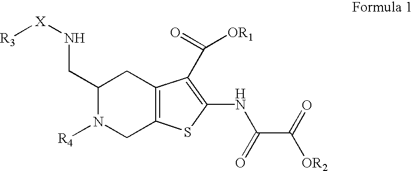

wherein

X is —C(O)— or —S(O)₂—;

R₁ and R₂ are independently hydrogen or a functional group that can be converted to hydrogen in vivo;

R₃ is hydrogen, C₁-C₆alkyl, H₂N—R₃₅—, C₂-C₆alkenyl, C₂-C₆alkynyl, aryl, aryl-R₁₀—, aryl-N(R₃₅)—, aryl-R₁₁—N(R₃₆)—, C₁-C₆alkyloxy or aryl-R₁₃—O— wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, which aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or R₃₈—R₂₃—C(O)—N(R₂₄)—;

R₄ is hydrogen, C₁-C₆alkyl, aryl-R₂₆—, R₂₇—O—C(O)—, aryl-R₂₈—O—C(O)—, R₂₉—C(O)—O—R₃₀—O—C(O)— or aryl-R₃₁—C(O)—O—R₃₂—O—C(O)— wherein aryl is phenyl, naphthyl or thiophenyl, which aryl group is optionally substituted with halogen, nitro, cyano, trihalomethyl, R₃₉—, R₃₉—R₃₃—, C₁-C₆alkyloxy or R₃₉—R₃₄—O—;

and wherein R₅, R₇, R₈, R₉, R₁₀, R₁₁, R₁₃, R₁₄, R₁₅, R₂₃, R₂₆, R₂₈, R₃₀, R₃₁, R₃₂, R₃₃, R₃₄ and R₃₅ independently are C₁-C₆alkylene, wherein R₆, R₁₂, R₁₇, R₁₈, R₂₀, R₂₂, R₂₅, R₂₇ and R₂₉ independently are C₁-C₆alkyl and wherein R₁₆, R₁₉, R₂₁, R₂₄, R₃₅ and R₃₆, and R₃₇ independently are hydrogen or C₁-C₆alkyl;

R₃₈ and R₃₉ are independently phenyl, naphthyl or thiophenyl

or a salt thereof with a pharmaceutically acceptable acid or base, or any optical isomer or mixture of optical isomers, including a racemic mixture, or any tautomeric form.

In another embodiment R₁ and R₂ are independently hydrogen, C₁-C₆alkyl, aryl-R₅—, R₆—C(O)—O—R₇— or aryl-R₈—C(O)—O—R₉— wherein aryl is phenyl, naphthyl or thiophenyl, which aryl group is optionally substituted with halogen, nitro, trihalomethyl, C₁-C₆alkyl or C₁-C₆alkyloxy.

In another embodiment X is C(O).

In another embodiment X is S(O)₂.

In another embodiment R₁ and R₂ are independently hydrogen, C₁-C₆alkyl, aryl-R₅—, or R₆—C(O)—O—R₇—, wherein aryl is phenyl, naphthyl or thiophenyl, which aryl group is optionally substituted with halogen, nitro, trihalomethyl, C₁-C₆alkyl or C₁-C₆alkyloxy.

In another embodiment R₁ and R₂ are independently hydrogen or C₁-C₆alkyl.

In another embodiment R₁ and R₂ are hydrogen.

In another embodiment R₃ is C₁-C₆alkyl, C₂-C₆alkenyl, C₂-C₆alkynyl, aryl, aryl-R₁₀—, C₁-C₆alkyloxy, H₂N—R₃₅— or aryl-R₁₃—O— wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, which aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or R₃₈—R₂₃—C(O)—N(R₂₄)—.

In another embodiment R₃ is C₁-C₆alkyl, C₂-C₆alkenyl, aryl, H₂N—R₃₅— or aryl-R₁₃—O— wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, which aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—R₃₈—N(R₁₆)—, R₁₈—C(O)—N(R₁₉)—, R₃₈—C(O)—N(R₂₁)— or R₃₈—R₂₃—C(O)—N(R₂₄)—.

In another embodiment R₃ is C₁-C₆alkyl or aryl, wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, which aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁ g)—, R₃₈—C(O)—N(R₂₁)— or R₃₈—R₂₃—C(O)—N(R₂₄)—.

In another embodiment R₃ is aryl, wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, which aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁ g)—, R₃₈—C(O)—N(R₂₁)— or

R₃₈—R₂₃—C(O)—N(R₂₄)—.

In another embodiment the aryl of R₃ is phenyl, biphenyl, naphthyl, 1,2,3-triazolyl, quinolyl or indolyl.

In another embodiment the aryl of R₃ is phenyl, naphthyl, or indolyl.

In another embodiment the aryl of R₃ is phenyl.

In another embodiment the aryl of R₃ is substituted by halogen, hydroxy, C₁-C₆alkyl, R₃₇-aryl-, R₃₈—, R₃₈—R₁₄—, C₁-C₆alkyloxy, R₃₈—O—, R₃₈—R₁₅—O—, R₁₈—C(O)—N(R₁₉)—, or R₃₈—C(O)—N(R₂₁)—.

In another embodiment the aryl of R₃ is substituted by hydroxy, C₁-C₆alkyl, R₃₈—, R₃₈—R₁₄—, C₁-C₆alkyloxy, R₃₈—O—, R₁₈—C(O)—N(R₁₉)—, or R₃₈—C(O)—N(R₂₁)—.

In another embodiment R₃₈ is phenyl or thiophenyl.

In another embodiment R₄ is hydrogen, C₁-C₆alkyl, aryl-R₂₆—, R₂₉—C(O)—O—R₃₀—O—C(O)— or aryl-R₃₁—C(O)—O—R₃₂—O—C(O)— wherein aryl is phenyl, naphthyl or thiophenyl, which aryl group is optionally substituted with halogen, nitro, cyano, trihalomethyl, R₃₉—, R₃₉—R₃₃—, C₁-C₆alkyloxy or R₃₉—R₃₄—O—.

In another embodiment R₄ is hydrogen, C₁-C₆alkyl, or aryl-R₂₆— wherein aryl is phenyl, naphthyl or thiophenyl, which aryl group is optionally substituted with halogen, nitro, cyano, trihalomethyl, R₃₉—, R₃₉—R₃₃—, C₁-C₆alkyloxy or R₃₉—R₃₄—O—.

In another embodiment R₄ is hydrogen or aryl-R₂₆— wherein aryl is phenyl, naphthyl or thiophenyl, which aryl group is optionally substituted with halogen, nitro, cyano, trihalomethyl, R₃₉—, R₃₉—R₃₃—, C₁-C₆alkyloxy or R₃₉—R₃₄—O—.

In another embodiment R₄ is hydrogen.

In another embodiment R₄ is aryl-R₂₆— wherein aryl is phenyl, naphthyl or thiophenyl, which aryl group is optionally substituted with halogen, nitro, cyano, trihalomethyl, R₃₉—, R₃₉—R₃₃—, C₁-C₆alkyloxy or R₃₉—R₃₄—O—.

In another embodiment the aryl of R₄ is phenyl.

In another embodiment the aryl of R₄ is substituted by R₃₉—.

In another embodiment R₃₉ is C₁-C₆alkyloxy.

In another embodiment R₃₉ is methoxy.

Another aspect of the invention is compounds according to the invention that act as inhibitors of Protein Tyrosine Phosphatases.

Another aspect of the invention is a pharmaceutical composition comprising a compound of the invention or a pharmaceutically acceptable salt thereof with a pharmaceutically acceptable acid or base, or any optical isomer or mixture of optical isomers, including a racemic mixture, or any tautomeric form together with one or more pharmaceutically acceptable carriers or diluents.

Another aspect of the invention is a pharmaceutical composition suitable for treating type 1 diabetes, type 2 diabetes, impaired glucose tolerance, insulin resistance or obesity comprising a compound of the invention or a pharmaceutically acceptable salt thereof with a pharmaceutically acceptable acid or base, or any optical isomer or mixture of optical isomers, including a racemic mixture, or any tautomeric form together with one or more pharmaceutically acceptable carriers or diluents.

Another aspect of the invention is a pharmaceutical composition suitable for treating immune dysfunctions including autoimmunity, diseases with dysfunctions of the coagulation system, allergic diseases, osteoporosis, proliferative disorders including cancer and psoriasis, diseases with decreased or increased synthesis or effects of growth hormone, diseases with decreased or increased synthesis of hormones or cytokines that regulate the release of/or response to growth hormone, diseases of the brain including Alzheimer's disease and schizophrenia, and infectious diseases comprising a compound of the invention or a pharmaceutical acceptable salt thereof with a pharmaceutically acceptable acid or base, or any optical isomer or mixture of optical isomers, including a racemic mixture, or any tautomeric form together with one or more pharmaceutically acceptable carriers or diluents. The composition may be in the form of an oral dosage unit or parenteral dosage unit.

The pharmaceutical composition may said may contain a compound of the invention such that it is administered as a dose in a range from about 0.05 to 1000 mg, preferably from about 0.1 to 500 mg and especially in the range from 50 to 200 mg per day.

Another aspect of the invention is a compound of the invention or a pharmaceutically acceptable salt thereof with a pharmaceutically acceptable acid or base, or any optical isomer or mixture of optical isomers, including a racemic mixture, or any tautomeric form for therapeutical use in the treatment or prevention of type 1 diabetes, type 2 diabetes, impaired glucose tolerance, insulin resistance or obesity.

Another aspect of the invention is a compound of the invention or a pharmaceutically acceptable salt thereof with a pharmaceutically acceptable acid or base, or any optical isomer or mixture of optical isomers, including a racemic mixture, or any tautomeric form for therapeutical use in the treatment or preventing of immune dysfunctions including autoimmunity, diseases with dysfunctions of the coagulation system, allergic diseases, osteoporosis, proliferative disorders including cancer and psoriasis, diseases with decreased or increased synthesis or effects of growth hormone, diseases with decreased or increased synthesis of hormones or cytokines that regulate the release of/or response to growth hormone, diseases of the brain including Alzheimer's disease and schizophrenia, and infectious diseases.

Another aspect of the invention is the use of a compound of the invention or a pharmaceutically acceptable salt thereof with a pharmaceutically acceptable acid or base, or any optical isomer or mixture of optical isomers, including a racemic mixture, or any tautomeric form as a medicament.

Another aspect of the invention is the use of a compound of the invention for preparing a medicament.

Another aspect of the invention is the use of a compound of the invention or a pharmaceutically acceptable salt thereof with a pharmaceutically acceptable acid or base, or any optical isomer or mixture of optical isomers, including a racemic mixture, or any tautomeric form for the preparation of a medicament suitable for the treatment or preventing of type 1 diabetes, type 2 diabetes, impaired glucose tolerance, insulin resistance or obesity.

Another aspect of the invention is the use of a compound of the invention or a pharmaceutically acceptable salt thereof with a pharmaceutically acceptable acid or base, or any optical isomer or mixture of optical isomers, including a racemic mixture, or any tautomeric form for the preparation of a medicament suitable for the treatment or preventing of immune dysfunctions including autoimmunity, diseases with dysfunctions of the coagulation system, allergic diseases, osteoporosis, proliferative disorders including cancer and psoriasis, diseases with decreased or increased synthesis or effects of growth hormone, diseases with decreased or increased synthesis of hormones or cytokines that regulate the release of/or response to growth hormone, diseases of the brain including Alzheimer's disease and schizophrenia, and infectious diseases.

Another aspect of the invention is a method of treating type 1 diabetes, type 2 diabetes, impaired glucose tolerance, insulin resistance or obesity comprising administering to a subject in need thereof an effective amount of a compound of the invention.

Another aspect of the invention is a method of treating immune dysfunctions including autoimmunity, diseases with dysfunctions of the coagulation system, allergic diseases, osteoporosis, proliferative disorders including cancer and psoriasis, diseases with decreased or increased synthesis or effects of growth hormone, diseases with decreased or increased synthesis of hormones or cytokines that regulate the release of/or response to growth hormone, diseases of the brain including Alzheimer's disease and schizophrenia, and infectious diseases comprising administering to a subject in need thereof an effective amount of a compound of the invention to said subject.

Another aspect of the invention is a process for the manufacture of a medicament, particular to be used in the treatment or prevention of type 1 diabetes, type 2 diabetes, impaired glucose tolerance, insulin resistance or obesity which process comprising bringing a compound of the invention or a pharmaceutically acceptable salt thereof into a galenic dosage form.

Another aspect of the invention is a process for the manufacture of a medicament, particular to be used in the treatment or prevention of immune dysfunctions including autoimmunity, diseases with dysfunctions of the coagulation system, allergic diseases, osteoporosis, proliferative disorders including cancer and psoriasis, diseases with decreased or increased synthesis or effects of growth hormone, diseases with decreased or increased synthesis of hormones or cytokines that regulate the release of/or response to growth hormone, diseases of the brain including Alzheimer's disease and schizophrenia, and infectious diseases which process comprising bringing a compound of the invention or a pharmaceutically acceptable salt thereof into a galenic dosage form.

Another aspect of the invention is a method for preparing a compound of formula 1, characterized in

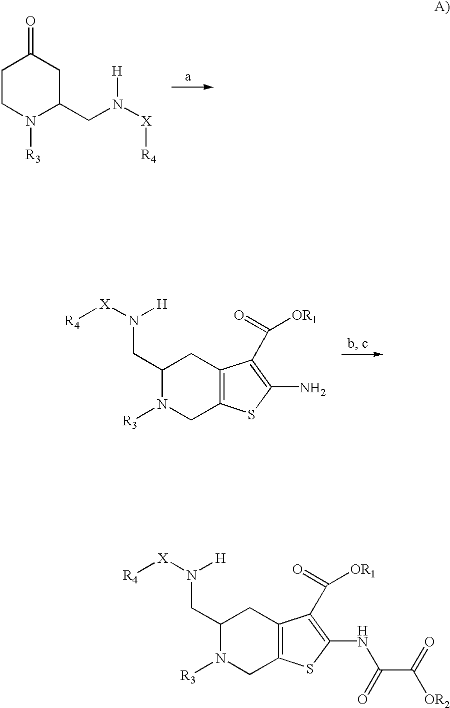

a) NC—CH₂—COOR₁, sulphur, morpholine or triethylamine, and EtOH; b) R₂—O—C(O)—C(O)-imidazol-1-yl, THF; c) 25% TFA/CH₂Cl₂; wherein R₁, R₂, R₃, R₄ and X are defined above.

The reaction step a) in Method A gives a mixture of regioisomers which can be separated by use of column chromatography known to thus skilled in the art.

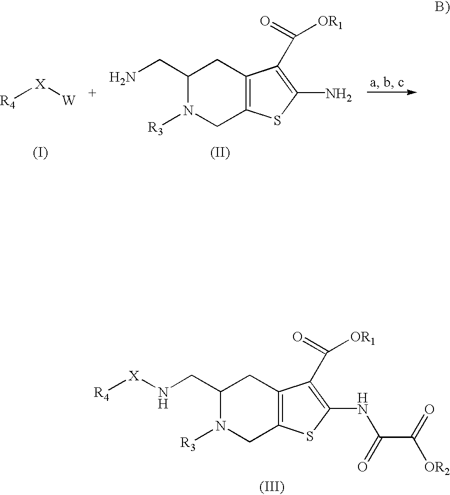

a) By allowing an activated carboxylic acid or sulfonic acid (I); wherein X is —C(CO)— and W is —OH, —OSO₂Me, halogen, R₄COO— or X is —SO₂— and W is chloride, a substituted 5-aminomethyl-tetrahydro-thieno[2,3-c]pyridine (II) to react under conditions known to thus skilled in the art which favour amide or sulfon amide bond formation followed by b) R₂—O—C(O)—C(O)-imidazol-1-yl, THF and c) 25% TFA/CH₂Cl₂; to yield (III) wherein R₁, R₂, R₃, and R₄ are defined above;

Pharmacological Methods

The compounds are evaluated for biological activity with a truncated form of PTP1B (corresponding to the first 321 amino acids), which was expressed in E. coli and purified to apparent homogeneity using published procedures well-known to those skilled in the art. The enzyme reactions are carried out using standard conditions essentially as described by Burke et al. (Biochemistry 35; 15989-15996 (1996)). The assay conditions are as follows. Appropriate concentrations of the compounds of the invention are added to the reaction mixtures containing different concentrations of the substrate, p-nitrophenyl phosphate (range: 0.16 to 10 mM—final assay concentration). The buffer used was 100 mM sodium acetate pH 5.5, 50 mM sodium chloride, 0.1% (w/v) bovine serum albumin and 5 mM dithiothreitol (total volume 100 ml). The reaction was started by addition of the enzyme and carried out in microtiter plates at 25° C. for 60 minutes. The reactions are stopped by addition of NaOH. The enzyme activity was determined by measurement of the absorbance at 405 nm with appropriate corrections for absorbance at 405 nm of the compounds and p-nitrophenyl phosphate. The data are analyzed using nonlinear regression fit to classical Michaelis Menten enzyme kinetic models. Inhibition is expressed as Ki values in μM. The results of representative experiments are shown in Table 1.

TABLE 1
Inhibition of classical PTP1B
by compounds of the invention

| Example | PTP1B |
|---|---|
| no. | Kᵢ values (μM) |
| 1 | 0.52 |
| 2 | 100 |
| 3 | 4.3 |
| 4 | 3.0 |
| 5 | 0.25 |
| 6 | 20.0 |
| 9 | 0.22 |
| 10 | 0.34 |
| 11 | 1.5 |
| 14 | 0.67 |
| 16 | 20 |
| 17 | 1.5 |
| 18 | 0.68 |
| 19 | 0.95 |
| 20 | 0.27 |
| 21 | 0.16 |
| 22 | 3.2 |
| 23 | 0.15 |
| 24 | 0.22 |
| 25 | 0.21 |
| 27 | 0.2 |
| 28 | 3.5 |
| 29 | 0.13 |

The Synthesis of the Compounds

In accordance with one aspect of the invention, the compounds of the invention are prepared as illustrated in the following reaction scheme:

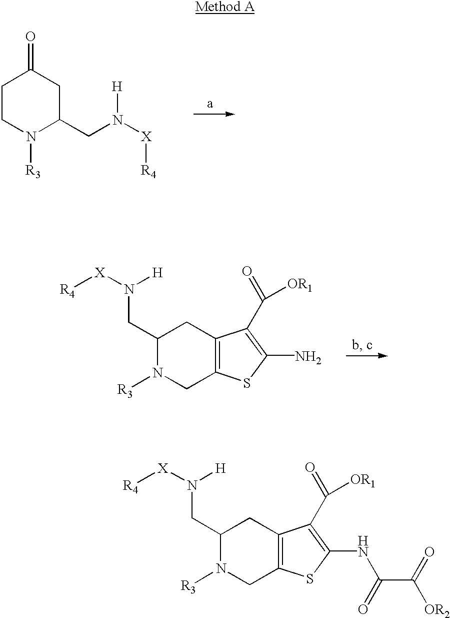

a) NC—CH₂—COOR₁, sulphur, morpholine or triethylamine, and ethanol; b) R₂—O—C(O)—C(O)-imidazol-1-yl, tetrahydrofurane; c) 25% trifluoroacetic acid/dichloromethane; wherein R₁, R₂, R₃, R₄ and X are defined above.

The reaction step a) in Method A gives a mixture of regioisomers which can be separated by use of column chromatography known to thus skilled in the art. Method B

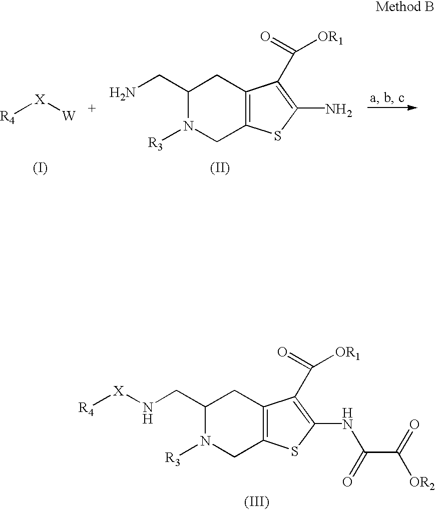

a) By allowing an activated carboxylic acid or sulfonic acid (I); wherein X is —C(CO)— and W is —OH, —OSO₂Me, halogen, R₄COO— or X is —SO₂— and W is chloride, a substituted 5-aminomethyl-tetrahydro-thieno[2,3-c]pyridine (II) to react under conditions known to thus skilled in the art which favour amide or sulfon amide bond formation followed by b) R₂—O—C(O)—C(O)-imidazol-1-yl, tetrahydrofuran and c) 25% trifluoroacetic acid/dichloromethane; to yield (III) wherein R₁, R₂, R₃, and R₄ are defined above.

Pharmacological Preparations

For the above indications the dosage will vary depending on the compound of the invention employed, on the mode of administration and on the therapy desired. However, in general, satisfactory results are obtained with a dosage of from about 0.5 mg to about 1000 mg, preferably from about 1 mg to about 500 mg of compounds of the invention, conveniently given from 1 to 5 times daily, optionally in sustained release form. Usually, dosage forms suitable for oral administration comprise from about 0.5 mg to about 1000 mg, preferably from about 1 mg to about 500 mg of the compounds of the invention admixed with a pharmaceutical carrier or diluent.

The compounds of the invention may be administered in a pharmaceutically acceptable acid addition salt form or where possible as a metal or a C₁₋₆-alkylammonium salt. Such salt forms exhibit approximately the same order of activity as the free acid forms.

This invention also relates to pharmaceutical compositions comprising a compound of the invention or a pharmaceutically acceptable salt thereof and, usually, such compositions also contain a pharmaceutical carrier or diluent. The compositions containing the compounds of this invention may be prepared by conventional techniques and appear in conventional forms, for example capsules, tablets, solutions or suspensions.

The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water.

Similarly, the carrier or diluent may include any time delay material known to the art, such as glyceryl monostearate or glyceryl distearate, alone or mixed with a wax.

If a solid carrier for oral administration is used, the preparation can be tablefted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatin capsule or sterile injectable liquid such as an aqueous or non-aqueous liquid suspension or solution.

Generally, the compounds of this invention are dispensed in unit dosage form comprising 10-200 mg of active ingredient in or together with a pharmaceutically acceptable carrier per unit dosage.

The dosage of the compounds according to this invention is 1-500 mg/day, e.g. about 100 mg per dose, when administered to patients, e.g. humans, as a drug.

A typical tablet that may be prepared by conventional tabletting techniques contains

| Core: | | | |
|---|---|---|---|
| Active compound (as free compound | | 100 | mg |
| or salt thereof) | | | |
| Colloidal silicon dioxide (Areosil ®) | | 1.5 | mg |
| Cellulose, microcryst. (Avicel ®) | | 70 | mg |
| Modified cellulose gum (Ac-Di-Sol ®) | | 7.5 | mg |
| Magnesium stearate | | | |
| Coating: | | | |
| HPMC | approx. | 9 | mg |
| *Mywacett ® 9-40 T | approx. | 0.9 | mg |

*Acylated monoglyceride used as plasticiser for film coating.

The route of administration may be any route, which effectively transports the active compound to the appropriate or desired site of action, such as oral or parenteral e.g. rectal, transdermal, subcutaneous, intranasal, intramuscular, topical, intravenous, intraurethral, ophthalmic solution or an ointment, the oral route being preferred.

A number of procedures, well known to those skilled in the art, may be used to verify that the attached chemical groups have been removed or that the cyclic compound has been hydrolyzed after uptake in cells or mammals. An example, which is not intended in any way to limit the scope of the invention, is given in the following. A mammalian cell line, which can be obtained from the American Tissue Type Collection or other similar governmental or commercial sources, is incubated with said modified compound. After incubation at conditions well known to those skilled in the art, the cells are washed appropriately, lysed and the lysate is isolated. Appropriate controls, well known to those skilled in the art, must be included. A number of different procedures, well known to those skilled in the art, may in turn be used to extract and purify said compound from said lysate. Said compound may or may not retain the attached chemical group or said cyclic compound may or may not have been hydrolyzed. Similarly, a number of different procedures—well known to those skilled in the art—may be used to structurally and chemically characterize said purified compound. Since said purified compound has been isolated from said cell lysate and hence has been taken up by said cell line, a comparison of said structurally and chemically characterized compound with that of the original unmodified compound (i.e. without said attached chemical group or said non-cyclic compound) will immediately provide those skilled in the art information on whether the attached chemical group as been removed in the cell or if the cyclic compound has been hydrolyzed. As a further analysis, said purified compound may be subjected to enzyme kinetic analysis as described in detail in the present invention. If the kinetic profile is similar to that of the original compound without said attached chemical group, but different from said modified compound, this confirms that said chemical group has been removed or said cyclic compounds has been hydrolyzed. Similar techniques may be used to analyze compounds of the invention in whole animals and mammals.

### EXAMPLES

The process for preparing compounds of Formula 1 and preparations containing them is further illustrated in the following examples, which, however, are not to be construed as limiting.

Hereinafter, TLC is thin layer chromatography, CDCl₃ is deuterio chloroform, CD₃OD is tetradeuterio methanol and DMSO-d₆ is hexadeuterio dimethylsulfoxide. The structures of the compounds were confirmed by either elemental analysis or NMR, where peaks assigned to characteristic protons in the title compounds are presented where appropriate. ¹H NMR shifts (δ_{H}) are given in parts per million (ppm) down field from tetramethylsilane as internal reference standard. M.p.: is melting point and is given in ° C. and is not corrected. Column chromatography was carried out using the technique described by W. C. Still et al., J. Org. Chem. 43:2923 (1978) on Merck silica gel 60 (Art. 9385). HPLC analyses are performed using 5 μm C18 4×250 mm column eluted with various mixtures of water and acetonitrile, flow=1 ml/min, as described in the experimental section.

Compounds used as starting material are either known compounds or compounds, which can readily be prepared by methods known per se.

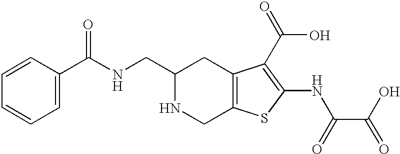

5-(Benzoylamino-methyl)-2-(oxalyl-amino)-4,5,6,7-tetrahydro-thieno[2,3-c]Pyridine-3-carboxylic Acid

To a solution of 4-methoxybenzyl amine (22.63 ml, 0.173 mol), 4 A molecular sieves (18 g), and dichloromethane (400 ml) was added ethyl glyoxalate (38.86 ml (50% solution in toluene), 0.19 mol). After stirring for 1 hr, the solution was cooled to 0° C. and trifluoroacetic acid (13.34 ml, 0.173 mol) was added via syringe. After 10 min, boron trifluoride etherate (21.94 ml, 0.173 mol) was added via syringe and the reaction mixture allowed to stir for 10 min. A solution of 2-trimethylsilyloxy-1,3-butadiene (25 g, 0.173 mol) in dichloromethane (30 ml) was added dropwise over a 5 min period. The reaction mixture was allowed to stir at 0° C. for 4 hours, at which time it was quenched with saturated sodium bicarbonate (100 ml). The mixture was filtered and the organic layer washed with saturated sodium bicarbonate (3×300 ml) and water (500 ml). The organic layer was dried (MgSO₄), filtered and evaporated in vacuo. The residue (51 g) was subjected to flash chromatography using a mixture of hexanes/ethyl acetate (60:40) as eluent which afforded 16.5 g (33%) of 4-oxo-1-(4-methoxy-benzyl)-piperidine-2-carboxylic acid ethyl ester as an oil.

¹H-NMR (CDCl₃) δ 7.28 (d, 2H, J=8.4 Hz), 6.86 (d, 2H, J=8.4 Hz), 4.20 (dq, 2H, J=7.2 and J=1.8 Hz), 3.97 (s, 3H), 3.70 (d, 2H, J=3.6 Hz), 3.01 (ddd, 1H, J=15.3, J=8.7 and J=5.4 Hz), 2.88-2.81 (m, 1H), 2.56 (dd, 1H, J=9.9 and J=6 Hz), 2.45-2.40 (m, 1H), 1.29 (t, 3H, J=7.2 Hz);

LC-MS: Rₜ=1.54, m/z: 292.3 [M+1]⁺.

To a solution of 4-oxo-1-(4-methoxy-benzyl)-piperidine-2-carboxylic acid ethyl ester (26.3 g, 0.09 mmol) in absolute ethanol (500 ml) was added tert-butylcyanoacetate (16.6 g, 0.12 mol), sulfur (3.18 g, 0.099 mol), and morpholine (15.7 ml, 0.18 mol). The reaction was placed in a 50° C. oil bath and stirred under nitrogen atmosphere for 18 hours. The mixture was cooled to ambient temperature, concentrated in vacuo, and the residue purified by silica gel chromatography using a gradient of ethyl acetate/hexane, (10% to 20% gradient) as eluent in two portions, which afforded a mixture of 2-amino-6-(4-methoxy-benzyl)-4,5,6,7-tetrahydro-thieno[2,3-c]pyridine-3,5-dicarboxylic acid 3-tert-butyl ester 5-ethyl ester and 2-amino-6-(4-methoxy-benzyl)-4,5,6,7-tetrahydro-thieno[2,3-c]pyridine-3,7-dicarboxylic acid 3-tert-butyl ester 7-ethyl ester 16.4 g (41%).

¹H-NMR (400 MHz, CDCl₃) δ 7.28 (d, J=8 Hz, 2H), 6.86 (d, J=8 Hz, 2H), 6.00-5.91 (2s, 2H), 4.24-4.13 (m, 2H), 3.83-2.64 (m, 7H), 3.80 (s, 3H), 1.54-1.52 (2s, 9H)

LC-MS: Rₜ=3.01 min, m/z: 447.2 [M+H]⁺.

A mixture of 2-amino-6-(4-methoxy-benzyl)-4,5,6,7-tetrahydro-thieno[2,3-c]pyridine-3,5-dicarboxylic acid 3-tert-butyl ester 5-ethyl ester and 2-amino-6-(4-methoxy-benzyl)-4,5,6,7-tetrahydro-thieno[2,3-c]pyridine-3,7-dicarboxylic acid 3-tert-butyl ester 7-ethyl ester (16.4 g, 36.7 mmol) was dissolved in anhydrous tetrahydrofuran (250 ml), treated with lithium borohydride (3.99 g, 184 mmol) and heated in a oil bath at 45° C. for 16 hours. TLC analysis showed that the reaction was incomplete. Additional lithium borohydride (0.8 g, 36.7 mmol) was added and the reaction stirred at 50° C. for an additional 6 hours. The solution was cooled to ambient temperature and neutralized by addition of 1N hydrochloric acid until no bubbling was observed and final pH=7 was obtained. The precipitate was filtratred off and the filtrate extracted with ethyl acetate (3×100 ml). The combined organic phases were washed with brine (100 ml), dried (MgSO₄), and the solvent evaporated in vacuo affording 14.4 g of a crude mixture of 2-amino-5-hydroxymethyl-6-(4-methoxy-benzyl)-4,5,6,7-tetrahydro-thieno[2,3-c]pyridine-3-carboxylic acid tert-butyl ester and 2-amino-7-hydroxymethyl-6-(4-methoxy-benzyl)-4,5,6,7-tetrahydro-thieno[2,3-c]pyridine-3-carboxylic acid tert-butyl ester as a solid.

¹H-NMR (400 MHz, CDCl₃) δ 7.23-7.19 (m, 2H), 6.87-6.82 (m, 2H), 5.99-5.91 (2s, 2H), 3.80 (s, 3H), 3.79-2.40 (m, 9H), 1.56-1.52 (2s, 9H)

LC-MS: Rₜ=2.50 min, m/z: 405.4 [M+H]⁺.

A solution of 2-amino-5-hydroxymethyl-6-(4-methoxy-benzyl)-4,5,6,7-tetrahydro-thieno[2,3-c]pyridine-3-carboxylic acid tert-butyl ester and 2-amino-7-hydroxymethyl-6-(4-methoxy-benzyl)-4,5,6,7-tetrahydro-thieno[2,3-c]pyridine-3-carboxylic acid tert-butyl ester (14.4 g, 35.6 mmol), phthalimide (5.76 g, 39.2 mmol), and triphenyl phosphine (10.3 g, 39.2 mmol) was prepared in anhydrous tetrahydrofuran (250 ml) and stirred under nitrogen with cooling in an ice bath. Diethyl azadicarboxylate (5.6 ml, 35.6 mmol) was added dropwise. The reaction was allowed to warm to ambient temperature overnight. The volatiles were removed and the crude mixture dissolved in dichloromethane (50 ml) and filtered to remove excess phthalimide. The filtrate was concentrated in vacuo and the residue purified by silica gel chromatography in two portions using a gradient of ethyl acetate/hexane, (20% to 25% gradient) as eluent to give 7.1 g of 2-amino-5-(1,3-dioxo-1,3-dihydro-isoindol-2-ylmethyl)-6-(4-methoxy-benzyl)-4,5,6,7-tetrahydro-thieno[2,3-c]pyridine-3-carboxylic acid tert-butyl ester.

¹H-NMR (400 MHz, CDCl₃) δ 7.87-7.84 (m, 2H), 7.75-7.73 (m, 2H), 7.07 (d, 2H, J=8 Hz), 6.70 (d, 2H, J=8 Hz), 5.94 (s, 2H), 4.09-4.05 (m, 1H), 3.84-3.73 (m, 2H), 3.76 (s, 3H), 3.66-3.43 (m, 4H), 2.89-2.83 (m, 1H), 2.67-2.61 (m, 1H), 1.53 (s, 9H)

LC-MS: Rₜ=3.03 min., m/z: 534.2 [M+H]⁺.

A mixture of 2-amino-5-(1,3-dioxo-1,3-dihydro-isoindol-2-ylmethyl)-6-(4-methoxy-benzyl)-4,5,6,7-tetrahydro-thieno[2,3-c]pyridine-3-carboxylic acid tert-butyl ester (7.1 g, 13.3 mmol) in anhydrous ethanol (95 ml) was placed in a sonicator for complete dissolution. To this solution was added anhydrous hydrazine (1.92 ml, 58.7 mmol) and the reaction placed in an 80° C. bath with stirring for 6 hours, then allowed to cool to ambient temperature and stirred for an additional 2 days. The precipitate was filtered off and washed with ethanol (2×25 ml). The filtrate was evaporated to dryness under reduced pressure, the residue dissolved in dichloromethane (50 ml), filtered, and the solvent removed in vacuo. All volatile materials were removed under vacuum (0.5×10⁻⁶ mm Hg) at 50° C. for 6 hours, which afforded 5.26 g (98%) of 2-amino-5-aminomethyl-6-(4-methoxy-benzyl)-4,5,6,7-tetrahydro-thieno[2,3-c]pyridine-3-carboxylic acid tert-butyl ester as a solid.

¹H-NMR (400 MHz, CDCl₃) δ 7.24 (d, 2H, J=8 Hz), 6.86 (d, 2H, J=8 Hz), 5.91 (s, 2H), 3.80 (s, 3H), 3.71-3.46 (m, 4H), 3.11-3.05 (m, 1H), 2.98-2.93 (m, 1H), 2.87-2.72 (m, 2H), 2.53-2.47 (m, 1H), 1.54 (s, 9H)

LC-MS: Rₜ=2.03 min, m/z: 404.2 [M+H]⁺.

2-Amino-5-aminomethyl-6-(4-methoxy-benzyl)-4,5,6,7-tetrahydro-thieno[2,3-c]pyridine-3-carboxylic acid tert-butyl ester (0.15 g, 0.37 mmol) was dissolved in a mixture of anhydrous dichloromethane (5 ml) and triethylamine (78 μl, 0.56 mmol). The flask was cooled in an ice-bath and stirred under nitrogen. Benzoyl chloride (43 μl, 0.37 mmol) was added dropwise and the reaction stirred for 5 minutes, then warmed to ambient temperature and stirred for an additional 16 hours. The solvent was removed in vacuo and the residue re-dissolved in ethyl acetate (20 ml). The organic phase was washed with 0.5 N hydrochloric acid (5 ml), saturated sodium bicarbonate (5 ml), and brine (5 ml). The solution was dried (MgSO₄), filtered, and concentrated in vacuo to give 0.18 g of 2-amino-5-(benzoylamino-methyl)-6-(4-methoxy-benzyl)-4,5,6,7-tetrahydro-thieno[2,3-c]pyridine-3-carboxylic acid tert-butyl ester.

¹H-NMR (300 MHz, CDCl₃) δ 7.77 (d, 2H, J=7 Hz), 7.51-7.42 (m, 3H), 7.21 (d, 2H, J=8 Hz), 7.00-6.98 (m, 1H), 6.84 (d, 2H, J=8 Hz), 5.96 (s, 2H), 3.78 (s, 3H), 3.75-3.50 (m, 5H), 3.40-3.23 (m, 2H), 2.92 (dd, 1H, J=18 Hz and J=6 Hz), 2.60 (dd, 1H, J=18 Hz and J=6 Hz), 1.55 (s, 9H)

LC-MS: Rₜ=1.29 min, m/z: 509 [M+H]⁺.

To a solution of crude 2-amino-5-(benzoylamino-methyl)-6-(4-methoxy-benzyl)-4,5,6,7-tetrahydro-thieno[2,3-c]pyridine-3-carboxylic acid tert-butyl ester (0.18 g, 0.35 mmol) in anhydrous dichloromethane (5 ml) was added imidazol-1-yl-oxo-acetic acid tert-butyl ester (0.20 g, 1.05 mmol). The reaction was stirred at ambient temperature for 16 hours. The solution was concentrated in vacuo and the residue purified by silica gel chromatography using a gradient of ethyl acetate/dichloromethane (5% to 10% gradient) as eluent, which afforded 0.18 g of 5-(benzoylamino-methyl)-2-(tert-butoxyoxalyl-amino)-6-(4-methoxy-benzyl)-4,5,6,7-tetrahydro-thieno[2,3-c]pyridine-3-carboxylic acid tert-butyl ester. The overall yield for the first two steps was 77%.

¹H-NMR (300 MHz, CDCl₃) δ 12.54 (s, 1H), 7.76 (d, 2H, J=7 Hz), 7.51-7.42 (m, 3H), 7.19 (d, 2H, J=8 Hz), 6.99-6.96 (m, 1H), 6.83 (d, 2H, J=8 Hz), 3.87-3.68 (m, 4H), 3.77 (s, 3H), 3.58-3.53 (m, 1H), 3.40-3.29 (m, 2H), 2.99 (bd, 1H, J=18 Hz), 2.68 (bd, 1H, J=18 Hz), 1.60 (s, 9H)

LC-MS: Rₜ=3.57 min, m/z: 636 [M+H]⁺.

5-(Benzoylamino-methyl)-2-(tert-butoxyoxalyl-amino)-6-(4-methoxy-benzyl)-4,5,6,7-tetrahydro-thieno[2,3-c]pyridine-3-carboxylic acid tert-butyl ester (0.18 g, 0.283 mmol) was dissolved in a solution of 10% formic acid/methanol (4 ml). Palladium on activated carbon (10%, 90 mg) was added and the reaction stirred at ambient temperature for 24 hours. Another 70 mg of catalyst was added and the reaction stirred for an additional 72 hours. The solution was filtered through celite and the filter cake washed with hot methanol. The filtrate was concentrated in vacuo and the residue precipitated from hexanes. The solid material was filtered off and washed further with hexane affording 90 mg (62%) of 5-(benzoyl-amino-methyl)-2-(tert-butoxyoxalyl-amino)-4,5,6,7-tetrahydro-thieno[2,3-c]pyridine-3-carboxylic acid tert-butyl ester as a solid.

¹H-NMR (400 MHz, CDCl₃) δ 12.49 (s, 1H), 8.23 (bs, 1H), 7.97 (bs, 1H), 7.85 (d, 2H, J=7 Hz), 7.49-7.38 (m, 3H), 4.17-4.04 (m, 2H), 3.99-3.94 (m, 1H), 3.65-3.59 (m, 1H), 3.47-3.41 (m, 1H), 3.22 (d, 1H, J=16 Hz), 2.81 (dd, 1H, J=16 Hz and J=10 Hz), 1.61 (s, 9H), 1.59 (s, 9H)

LC-MS: Rₜ=1.26 min, m/z: 404 (loss of two t-butyl groups) [M+H]⁺.

5-(Benzoyl-amino-methyl)-2-(tert-butoxyoxalyl-amino)-4,5,6,7-tetrahydro-thieno[2,3-c]pyridine-3-carboxylic acid tert-butyl ester (60 mg, 0.12 mmol) was dissolved in a solution of 50% trifluoroacetic acid/dichloromethane (3 ml) and stirred at ambient temperature for 16 hours. Adding the trifluoroacetic acid solution dropwise to diethyl ether precipitated the product. The suspension was stirred for 1 hour, filtered and washed with diethyl ether, which afforded 36 mg (60%) of the title compound as a solid.

¹H-NMR (400 MHz, DMSO) δ 12.34 (bs, 1H), 9.24 (bs, 2H), 8.86 (s, 1H), 7.89 (d, 2H, J=7 Hz), 7.56-7.46 (m, 3H), 4.39-4.24 (m, 2H), 3.68-3.59 (m, 3H), 3.24-3.18 (m, 2H, partially obscured by water), 2.89-2.83 (m, 1H), 2.65-2.32 (m, 1H, partially obscured by DMSO)

LC-MS: Rₜ=0.63 min, m/z: 404 [M+H]⁺.

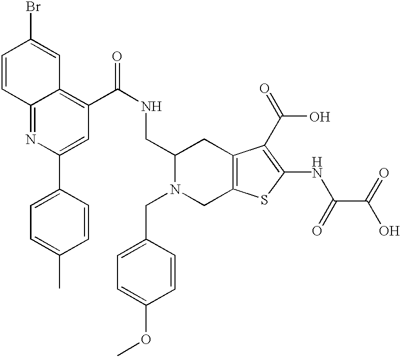

5-(((6-Bromo-2-p-tolyl-quinoline-4-carbonyl)amino)methyl)-6-(4-methoxy-benzyl)-2-(oxalyl-amino)-4,5,6,7-tetrahydro-thieno[2,3-c]pyridine-3-carboxylic Acid

The title compound was prepared in a similar way as described in Example 1 using 6-bromo-2-p-tolyl-quinoline-4-carboxylic acid and 2-amino-5-aminomethyl-6-(4-methoxy-benzyl)-4,5,6,7-tetrahydro-thieno[2,3-c]pyridine-3-carboxylic acid tert-butyl ester as the starting material. The hydrogenation step in Example I was omitted.

LC-MS: m/z: 745.2 [M+H]⁺

Calculated for C₃₆H₃₁N₄O₇SBr, C₂HF₃O₂;

C, 53.22%; H, 3.76%; N, 6.65%. Found:

C, 52.96%; H, 4.04%; N, 6.63%.

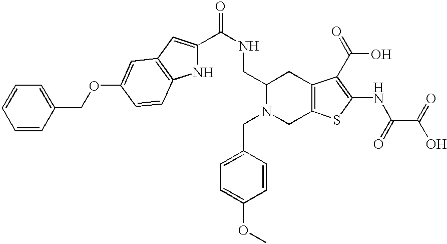

5-(((5-Benzyloxy-1H-indole-2-carbonyl)amino)methyl)-6-(4-methoxy-benzyl)-2-(oxalyl-amino)-4,5,6,7-tetrahydro-thieno[2,3-c]pyridine-3-carboxylic Acid

The title compound was prepared in a similar way as described in Example 1 using 5-benzyloxy-indole-2-carboxylic acid and 2-amino-5-aminomethyl-6-(4-methoxy-benzyl)-4,5,6,7-tetrahydro-thieno[2,3-c]pyridine-3-carboxylic acid tert-butyl ester as the starting material. The hydrogenation step in Example 1 was omitted.

LC-MS: m/z: 669.4 [M+H]⁺

Calculated for C₃₅H₃₂N₄O₈S, 0.5xC₂HF₃O₂, H₂O;

C, 58.14%; H, 4.68%; N, 7.53%. Found:

C, 58.13%; H, 4.61%; N, 7.50%.

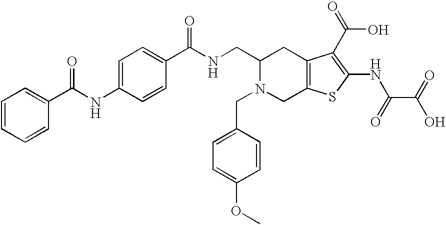

5-((4-Benzoylamino-benzoylamino)methyl)-6-(4-methoxy-benzyl)-2-oxalyl amino)-4,5,6,7-tetrahydro-thieno[2,3-c]pyridine-3-carboxylic Acid

The title compound was prepared in a similar way as described in Example 1 using 4-(benzoylamino)benzoic acid and 2-amino-5-aminomethyl-6-(4-methoxy-benzyl)-4,5,6,7-tetrahydro-thieno[2,3-c]pyridine-3-carboxylic acid tert-butyl ester as the starting material. The hydrogenation step in Example 1 was omitted.

LC-MS: m/z: 643.2 [M+H]⁺

Calculated for C₃₃H₃₀N₄O₈S, 2xC₂HF₃O₂;

C, 51.04%; H, 3.70%; N, 6.43%. Found:

C, 50.74%; H, 3.98%; N, 6.49%.

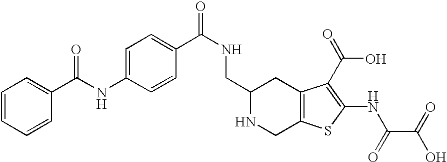

5-((4-Benzoylamino-benzoylamino)methyl)-2-(oxalyl-amino)-4,5,6,7-tetrahydro-thieno[2,3-c]pyridine-3-carboxylic Acid

The title compound was prepared in a similar way as described in Example 1 using 4-(benzoylamino)-benzoic acid and 2-amino-5-aminomethyl-6-(4-methoxy-benzyl)-4,5,6,7-tetrahydro-thieno[2,3-c]pyridine-3-carboxylic acid tert-butyl ester as the starting material.

LC-MS: m/z: 523.0 [M+H]⁺

Calculated for C₂₅H₂₂N₄O₇S, 2/3xC₂HF₃O₂, H₂O;

C, 51.80%; H, 3.96%; N, 9.18%. Found:

C, 51.99%; H, 4.32%; N, 8.94%.

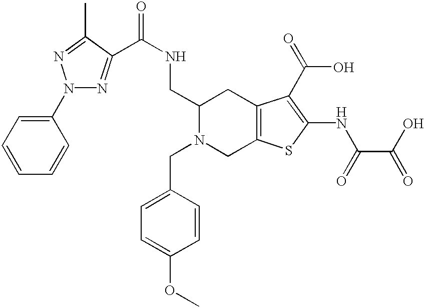

6-(4-Methoxy-benzyl)-5-(((5-methyl-2-phenyl-2H-[1,2,3]triazole-4-carbonyl)-amino)methyl)-2-(oxalyl-amino)-4,5,6,7-tetrahydro-thieno[2,3-c]pyridine-3-carboxylic Acid

The title compound was prepared in a similar way as described in Example 1 using 5-methyl-2-phenyl-2H-[1,2,3]triazole-4-carboxylic acid and 2-amino-5-aminomethyl-6-(4-methoxy-benzyl)-4,5,6,7-tetrahydro-thieno[2,3-c]pyridine-3-carboxylic acid tert-butyl ester as the starting material.

The hydrogenation step in Example 1 was omitted.

LC-MS: m/z: 605.2 [M+H]⁺

Calculated for C₂₉H₂₈N₆O₇S, 1.5xC₂HF₃O₂, H₂O;

C, 48.43%; H, 4.00%; N, 10.59%. Found:

C, 48.72%; H, 4.19%; N, 10.18%.

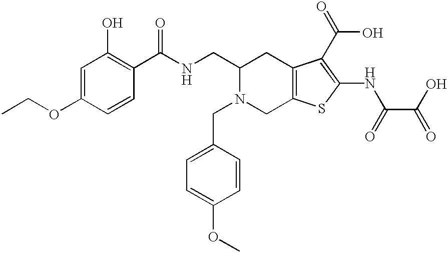

5-((4-Ethoxy-2-hydroxy-benzoylamino)methyl)-6-(4-methoxy-benzyl)-2-(oxalyl-amino)-4,5,6,7-tetrahydro-thieno[2,3-c]pyridine-3-carboxylic Acid

The title compound was prepared in a similar way as described in Example 1 using 2-hydroxy-4-ethoxybenzoic acid and 2-amino-5-aminomethyl-6-(4-methoxy-benzyl)-4,5,6,7-tetrahydro-thieno[2,3-c]pyridine-3-carboxylic acid tert-butyl ester as the starting material. The hydrogenation step in Example 1 was omitted.

LC-MS: m/z: 585.2 [M+H]⁺

Calculated for C₃₆H₃₁N₄O₇SBr, C₂HF₃O₂, 2/3xH₂O;

C, 48.19%; H, 4.24%; N, 5.44%. Found:

C, 47.99%; H, 4.26%; N, 5.27%.

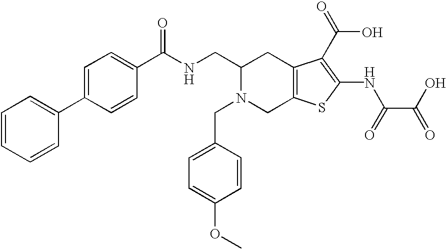

5-(((Biphenyl-4-carbonyl)amino)methyl)-6-(4-methoxy-benzyl)-2-(oxalyl-amino)-4,5,6,7-tetrahydro-thieno[2,3-c]pyridine-3-carboxylic Acid

The title compound was prepared in a similar way as described in Example 1 using 4-phenylbenzoic acid and 2-amino-5-aminomethyl-6-(4-methoxy-benzyl)-4,5,6,7-tetrahydro-thieno[2,3-c]pyridine-3-carboxylic acid tert-butyl ester as the starting material. The hydrogenation step in Example 1 was omitted.

LC-MS: m/z: 601.1 [M+H]⁺

Calculated for C₃₂H₂₉N₃O₇S, 2xC₂HF₃O₂, 2xH₂O;

C, 50.06%; H, 4.08%; N, 4.86%. Found:

C, 50.20%; H, 3.83%; N, 4.70%.

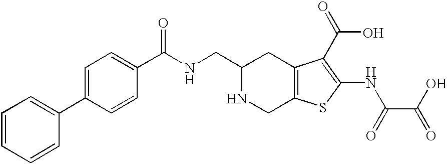

5-(((Biphenyl-4-carbonyl)amino)methyl)-2-(oxalyl-amino)-4,5,6,7-tetrahydro-thieno[2,3-c]pyridine-3-carboxylic Acid

The title compound was prepared in a similar way as described in Example 1 using 4-phenylbenzoic acid and 2-amino-5-aminomethyl-6-(4-methoxy-benzyl)-4,5,6,7-tetrahydro-thieno[2,3-c]pyridine-3-carboxylic acid tert-butyl ester as the starting material.

LC-MS: m/z: 480.2 [M+H]⁺

HPLC (A1): Rₜ=25.81 min.

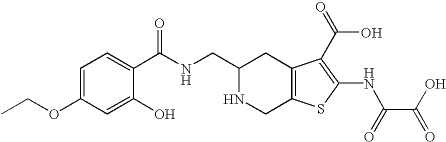

5-((4-Ethoxy-2-hydroxy-benzoylamino)methyl)-2-(oxalyl-amino)-4,5,6,7-tetrahydro-thieno[2,3-c]pyridine-3-carboxylic Acid

The title compound was prepared in a similar way as described in Example 1 using 2-hydroxy-4-ethoxybenzoic acid and 2-amino-5-aminomethyl-6-(4-methoxy-benzyl)-4,5,6,7-tetrahydro-thieno[2,3-c]pyridine-3-carboxylic acid tert-butyl ester as the starting material.

LC-MS: m/z: 464.1 [M+H]⁺

HPLC (A1): Rₜ=19.98 min.

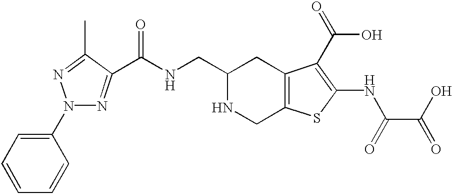

5-(((5-Methyl-2-phenyl-2H-[1,2,3]triazole-4-carbonyl)amino]methyl)-2-(oxalyl-amino)-4,5,6,7-tetrahydro-thieno[2,3-c]pyridin-3-carboxylic Acid

The title compound was prepared in a similar way as described in Example 1 using 5-methyl-2-phenyl-2H-[1,2,3]triazole-4-carboxylic acid and 2-amino-5-aminomethyl-6-(4-methoxy-benzyl)-4,5,6,7-tetrahydrothieno[2,3-c]pyridine-3-carboxylic acid tert-butyl ester as the starting material.

LC-MS: m/z: 485.2 [M+H]⁺

HPLC (A1): Rₜ=22.04 min

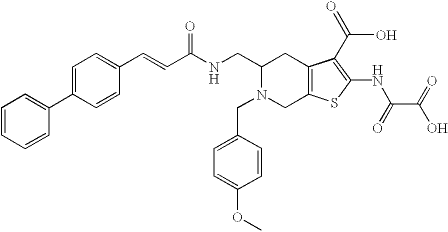

5-((3-Biphenyl-4-yl-acryloylamino)methyl)-6-(4-methoxy-benzyl)-2-(oxalyl-amino)-4,5,6,7-tetrahydro-thieno[2,3-c]pyridin-3-carboxylic Acid

The title compound was prepared in a similar way as described in Example 1 using 3-(biphenyl-4-yl)acrylic acid and 2-amino-5-aminomethyl-6-(4-methoxy-benzyl)-4,5,6,7-tetrahydro-thieno[2,3-c]pyridine-3-carboxylic acid tert-butyl ester as the starting material. The hydrogenation step in Example 1 was omitted.

LC-MS: m/z: 626.2 [M+H]⁺

HPLC (A1): Rₜ=35.60 min.

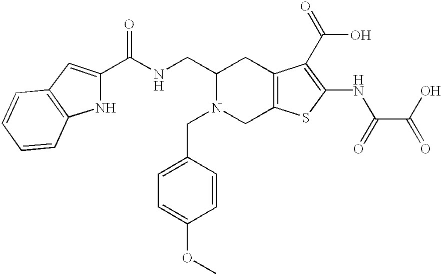

5-(((1H-Indole-2-carbonyl)-aminol-methyl}-6-(4-methoxy-benzyl)-2-(oxalyl-amino)-4,5,6,7-tetrahydro-thieno[2,3-c]pyridin-3-carboxylic Acid

The title compound was prepared in a similar way as described in Example 1 using indole-2-carboxylic acid and 2-amino-5-aminomethyl-6-(4-ethoxybenzyl)-4,5,6,7-tetrahydrothieno[2,3-c]pyridine-3-carboxylic acid tert-butyl ester as the starting material. The hydrogenation step in Example 1 was omitted.

LC-MS: m/z: 563.2 [M+H]⁺

HPLC (A1): Rₜ=27.59 min.

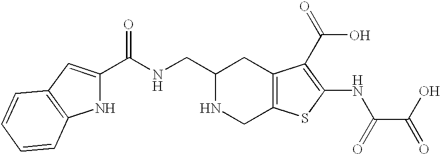

5-(((1H-Indole-2-carbonyl)-aminol-methyl}-2-(oxalyl-amino)-4,5,6,7-tetrahydro-thieno[2,3-c]pyridine-3-carboxylic Acid

The title compound was prepared in a similar way as described in Example 1 using 1H-indole-2-carboxylic acid and 2-amino-5-aminomethyl-6-(4-ethoxy-benzyl)-4,5,6,7-tetrahydrothieno[2,3-c]pyridine-3-carboxylic acid tert-butyl ester as the starting material.

LC-MS: m/z: 443.0 [M+H]⁺

HPLC (B1): Rₜ=19.67 min.

Calculated for C₂₀H₁₈N₄O₆S, 4xC₂HF₃O₂

C, 37.43%; H, 2.47%; N, 6.24%. Found:

C, 37.32%; H, 2.93%; N, 6.12%

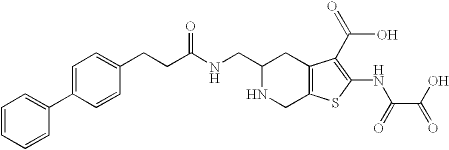

5-((3-Biphenyl-4-yl-propionylamino)methyl)-2-(oxalyl-amino)-4,5,6,7-tetrahydro-thieno[2,3-c]pyridine-3-carboxylic Acid

The title compound was prepared in a similar way as described in Example 1 using 3-(biphenyl-4-yl)acrylic acid and 2-amino-5-aminomethyl-6-(4-methoxy-benzyl)-4,5,6,7-tetrahydro-thieno[2,3-c]pyridine-3-carboxylic acid tert-butyl ester as the starting material.

LC-MS: m/z: 508,4 [M+H]⁺

HPLC (A1): Rₜ=22,23 min;

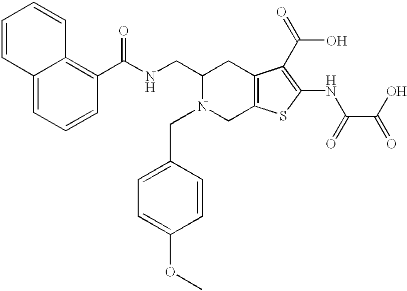

6-(4-Methoxy benzyl)-5-(((naphthalene-1-carbonyl)amino)methyl)-2-(oxalyl-amino)-4,5,6,7-tetrahydro-thieno[2,3-c]pyridine-3-carboxylic Acid

The title compound was prepared in a similar way as described in Example 1 using 1-naphthyl-carboxylic acid and 2-amino-5-aminomethyl-6-(4-ethoxy-benzyl)-4,5,6,7-tetrahydrothieno[2,3-c] pyridine-3-ca rboxylic acid tert-butyl ester as the starting material. The hydrogenation step in Example 1 was omitted.

LC-MS: m/z: 574.2 [M+H]⁺

HPLC (A1): Rₜ=27.36 min

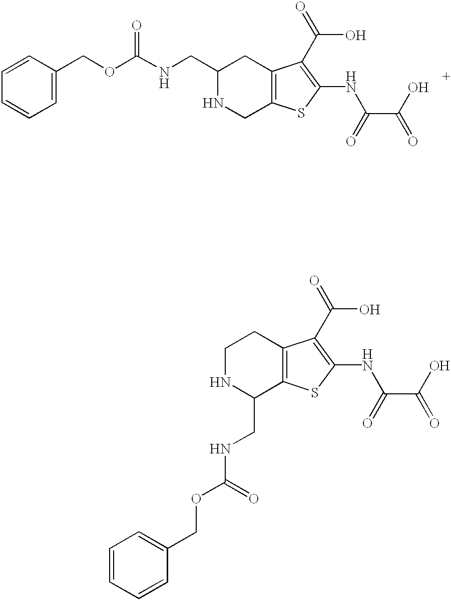

5-(Benzyloxycarbonylamino-methyl)-2-(oxalyl-amino)-4,5,6,7-tetrahydro-thieno[2,3-c]pyridine-3-carboxylic acid and 7-(benzyloxycarbonylamino-methyl)-2-(oxalyl-amino)-4,5,6,7-tetrahydro-thieno[2,3-c]pyridine-3-carboxylic Acid

In a 50 ml round-bottom flask, 2-(isoindole-1,3-dione-2-ylmethyl)-4-(2-spiro[1,3]dioxolane)-piperidine-1-carboxylic acid tert-butyl ester (543 mg, 1.35 mmol) was dissolved in 20% trifluoroacetic acid in dichloromethane (20 ml). After stirring in for 25 hours at room temperature, dichloromethane (30 ml) was added to dilute the solution. Solid sodium bicarbonate was then slowly added to neutralize the solution, which was then extracted with dichloromethane (3×40 ml). The combined organic extracts were dried (MgSO₄), filtered, and the solvent evaporated in vacuo, which afforded 385 mg (94%) of 2-(isoindole-1,3-dione-2-ylmethyl)-4-(2-spiro[1,3]dioxolane)-piperidine as a foam.

¹H-NMR (300 MHz, CDCl₃) δ 7.88-7.82 (m, 2H), 7.76-7.70 (m, 2H), 5.02-4.88 (bs, 1H), 4.66-4.55 (bs, 1H), 4.40-4.24 (bs, 1H), 4.08-3.08 (m, 8H), 2.26-1.75 (m, 2H).

In a 25 ml round-bottom flask, 2-(isoindole-1,3-dione-2-ylmethyl)-4-(2-spiro[1,3]dioxolane)-piperidine (385 mg, 1.27 mmol) was suspended in absolute ethanol (13 ml), flushed with nitrogen and sealed with a rubber septum. The reaction mixture was stirred at room temperature. Hydrazine (160 μI, 5.1 mmol) was added via syringe. The reaction mixture was heated to 80° C. for 6 hours and stirred at room temperature for 14 hours. The white precipitate was filtered off and washed with absolute ethanol. The filtrate was concentrated in vacuo affording an oil, which was dissolved in dichloromethane (10 ml) and re-concentrated in vacuo affording 214 mg (98%) of 2-aminomethyl-4-(2-spiro[1,3]dioxolane)-piperidine as a solid.

¹H-NMR (300 MHz, CDCl₃) δ 3.87 (s, 4H), 3.60-3.29 (bs, 2H), 3.27-3.01 (m, 2H), 2.98-2.79 (m, 2H), 2.78-2.67 (m, 1H), 1.95-1.62 (m, 3H), 1.61-1.45 (m, 1H).

In a 25 ml round-bottom flask, 2-aminomethyl-4-(2-spiro[1,3]dioxolane)-piperidine (553 mg, 3.21 mmol) in dichloromethane (10 ml) was flushed with nitrogen and sealed with a rubber septum. This solution was cooled to 0° C. before 95% benzyl chloroformate (550 μl, 3.9 mmol) was added dropwise. The solution was concentrated in vacuo and the residue was redissolved in 2N aqueous hydrochloric acid (12 ml) and heated to 50° C. with stirring for 15 hours. The reaction mixture was cooled to room temperature before dichloromethane (13 ml) and BOC-anhydride (11 ml, 10.4 g, 48 mmol) were added. Solid sodium bicarbonate was added slowly until the pH of the solution was 8. The bi-phasic mixture was stirred briskly at room temperaturet for 5 hours, extracted with dichloromethane (2×30 ml). The combined organic extracts were washed with brine (25 ml), dried (MgSO₄), filtered, and the solvent evaporated in vacuo. The residue was purified by silica gel chromatography using a mixture of hexanes/ethyl acetate (3:1), which afforded 430 mg (37%) of 2-(benzyloxycarbonyl-amino-methyl)-4-oxo-piperidine-1-carboxylic acid tert-butyl ester as an oil.

¹H-NMR (300 MHz, CDCl₃) δ 7.40-7.30 (bs, 5H), 5.09 (s, 2H), 4.74-4.63 (bs, 1H), 4.36-4.16 (bs, 1H), 3.50-3.10 (m, 3H), 2.66 (dd, 1H, J=16 Hz and J=7 Hz), 2.56-2.29 (m, 3H), 1.46 (s, 9H).

In a 50 ml round-bottom flask, 2-(benzyloxycarbonyl-amino-methyl)-4-oxo-piperidine-1-carboxylic acid tert-butyl ester (430 mg, 1.19 mmol) in absolute ethanol (15 ml) was flushed with nitrogen and sealed with a rubber septum. Sulfur (44 mg, 1.4 mmol) and tert-butyl cyano acetate (218 μl, 1.5 mmol) were added. The solution was cooled to 0° C. before morpholine (210 μl, 2.4 mmol) was added. Stirring was commenced as the reaction was heated to 50° C. for 20 hours. The cooled reaction mixture was concentration in vacuo and the residual oil was purified by silica gel chromatography using a mixture of hexanes/ethyl acetate (6:1) as eluent affording 474 mg (77%) of a 1:1 mixture of 2-amino-7-(benzyloxycarbonylamino-methyl)-4,7-dihydro-5H-thieno[2,3-c]pyridine-3,6-dicarboxylic acid di-tert-butyl ester and 2-amino-5-(benzyloxy-carbonylamino-methyl)-4,7-dihydro-5H-thieno[2,3-c]pyridine-3,6-dicarboxylic acid di-tert-butyl ester as a solids.

¹H-NMR (300 MHz, CDCl₃) δ 7.40-7.32 (m, 5H), 5.42-2.41 (m, 9H), 1.55 (s, 9H), 1.46 (s, 4.5H), 1.45 (s, 4.5H).

In a 10 ml scintillating vial a solution of 2-amino-7-(benzyloxycarbonylamino-methyl)-4,7-dihydro-5H-thieno[2,3-c]pyridine-3,6-dicarboxylic acid di-tert-butyl ester and 2-amino-5-(benzyloxy-carbonylamino-methyl)-4,7-dihydro-5H-thieno[2,3-c]pyridine-3,6-dicarboxylic acid di-tert-butyl ester (193 mg, 0.37 mmol) in dichloromethane (4 ml) was treated with imidazol-1-yl-oxo-acetic acid tert-butyl ester (520 mg, 2.7 mmol) in dichloromethane (2 ml). After stirring for 3 hours at room temperature, the reaction solution was concentrated in vacuo. The residue was purified by silica gel chromatography using a mixture of hexanes/ethyl acetate (6:1), which afforded 192 mg (77%) of a mixture of 5-(benzyloxy-carbonylamino-methyl)-2-(tert-butoxyoxalyl-amino)-4,7-dihydro-5H-thieno[2,3-c]pyridine-3,6-dicarboxylic acid di-tert-butyl ester and 7-(benzyloxy-carbonylamino-methyl)-2-(tert-butoxyoxalyl-amino)-4,7-dihydro-5H-thieno[2,3-c]pyridine-3,6-dicarboxylic acid di-tert-butyl ester as a foam.

LC-MS (APCI⁺) m/z: 646.3 [M+H]⁺;

In a 10 ml round-bottom flask, a mixture of 5-(benzyloxy-carbonylamino-methyl)-2-(tert-butoxyoxalyl-amino)-4,7-dihydro-5H-thieno[2,3-c]pyridine-3,6-dicarboxylic acid di-tert-butyl ester and 7-(benzyloxy-carbonylamino-methyl)-2-(tert-butoxy-oxalyl-amino)-4,7-di hydro-5H-thieno[2,3-c]pyridine-3,6-dicarboxylic acid d 1-tert-butyl ester (46 mg, 0.07 mmol) was dissolved in 30% trifluoroacetic acid in dichloromethane (4 ml). The solution was left open to the atmosphere without stirring. After 24 hours a precipitate was filtered off and washed with diethyl ether, affording 35 mg (90%) of the title compounds as a solid.

¹H-NMR (300 MHz, DMSO) δ 12.35 (bs, 1H), 9.28 (bs, 2H), 7.79 (s, 0.5H), 7.68 (s, 0.5H), 7.42-7.30 (m, 5H), 5.09 (s, 1H), 5.06 (s, 1H), 4.57 (bs, 0.5H), 4.30 (q, 1H, J=6 Hz), 3.60-2.70 (m partially obscured by water, 7H).

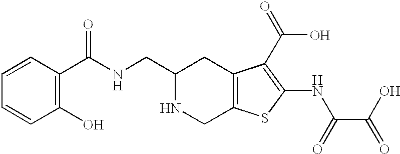

5-((2-Hydroxy-benzoylamino)-methyl)-2-(oxalyl-amino)-4,5,6,7-tetrahydro-thieno[2,3-c]pyridine-3-carboxylic Acid

Salicylic acid (1.25 g, 9.05 mmol) was dissolved in anhydrous N,N-dimethyl-formamide (15 ml) and placed under nitrogen atmosphere. Sodium hydride (0.76 g, 18.1 mmol) was added and the reaction stirred for 10 minutes, then benzyl bromide (4.3 ml, 36.2 mmol) was added. The reaction was stirred at ambient temperature for 4 days. The reaction mixture was diluted with ethyl acetate (75 ml) and washed with water (3×25 ml) followed by brine (15 ml). The organic layer was dried (MgSO₄), filtered, and the solvent evaporated in vacuo. The residue was purified by silica gel chromatography affording 2.61 g (90%) of 2-benzyloxy-benzoic acid benzyl ester as an oil.

¹H-NMR (400 MHz, CDCl₃) δ 7.86 (dd, 1H, J=8 Hz and J=2 Hz), 7.46-7.39 (m, 5H), 7.36-7.30 (m, 6H), 7.03-6.97 (m, 2H), 5.35 (s, 2H), 5.16 (s, 2H).

LC-MS: Rₜ=4.03 min, m/z: 319 [M+H]⁺.

2-Benzyloxy-benzoic acid benzyl ester (2.61 g, 8.15 mmol) was dissolved in a solution of methanol (15 ml) and tetrahydrofuran (7 ml). Sodium hydroxide (0.49 g, 12.22 mmol) was dissolved in water (2 ml) and added to the reaction mixture.

The reaction was stirred at ambient temperature for 24 hours, quenched with 6N hydrochloric acid to a final pH of 5. The mixture was evaporated to dryness in vacuo and the residue extracted into ethyl acetate (50 ml). The organic layer was extracted with 1N sodium hydroxide (30 ml) and the layers separated. The aqueous layer was washed with dichloromethane (2×10 ml), acidified with 6N hydrochloric acid and extracted with ethyl acetate (2×30 ml). The combined organic extracts were washed with brine (15 ml), dried (MgSO₄), filtered and the solvent evaporated in vacuo to give 1.71 g (92%) of 2-benzyloxy-benzoic acid as an oil

¹H-NMR (400 MHz, CDCl₃) δ 8.20 (dd, 1H, J=8 Hz and J=2 Hz), 7.58-7.53 (m, 1H), 7.45-7.40 (m, 5H), 7.17-7.12 (m, 2H), 5.30 (s, 2H)

LC-MS: Rₜ=1.21 min, m/z:=229 [M+H]⁺.

To a solution of 2-benzyloxy-benzoic acid (0.12 g, 0.53 mmol) and 1-(3-dimethylaminopropyl)-3-ethylcarbodiimide hydrochloride (0.10 g, 0.62 mmol) in anhydrous acetonitrile (5 ml) was added triethylamine (0.18 ml, 1.6 mmol), followed by 2-amino-5-aminomethyl-6-(4-methoxy-benzyl)-4,5,6,7-tetrahydro-thieno[2,3-c]pyridine-3-carboxylic acid tert-butyl ester (0.176 g, 0.44 mmol). The reaction was stirred under nitrogen atmosphere for 16 hours. The solvents were removed in vacuo and the residue partitioned between ethyl acetate (30 ml) and water (10 ml). The layers were separated and the organic phase washed with 1% hydrochloric acid (10 ml), saturated sodium bicarbonate (10 ml), and brine (10 ml). The organic layer was dried (MgSO₄), filtered, and the solvent evaporated in vacuo. The residue was purified by silica gel chromatography using a mixture of methanol/dichloromethane (1:19) as eluent, which afforded 82 mg (31%) of 2-amino-5-((2-benzyloxy-benzoylamino)methyl)-6-(4-methoxy-benzyl)-4,5,6,7-tetrahydro-thieno[2,3-c]pyridine-3-carboxylic acid tert-butyl ester as an oil.

¹H-NMR (300 MHz, CDCl₃) δ 8.43 (bs, 1H), 8.23 (d, 1H, J=7 Hz), 7.46-7.40 (m, 1H), 7.37-7.33 (m, 2H), 7.30-7.27 (m, 3H), 7.13-7.03 (m, 4H), 6.71 (d, 2H, J=8 Hz), 5.95 (s, 2H), 5.20 (s, 2H), 3.82-3.74 (m, 1H), 3.76 (s, 3H), 3.61-3.29 (m, 4H), 3.12-3.06 (m, 2H), 2.85-2.77 (m, 1H), 2.57-2.50 (m, 1H), 1.53 (s, 9H)

LC-MS: Rₜ=1.39 min, m/z: 614 [M+H]⁺.

To a solution of 2-amino-5-((2-benzyloxy-benzoylamino)methyl)-6-(4-methoxy-benzyl)-4,5,6,7-tetrahydro-thieno[2,3-c]pyridine-3-carboxylic acid tert-butyl ester (81 mg, 0.132 mmol) in anhydrous dichloromethane (4 ml) was added imidazol-1-yl-oxo-acetic acid tert-butyl ester (80 mg, 0.4 mmol). The reaction was stirred at ambient temperature for 3 hours and the solvent evaporated in vacuo. The residue was purified by silica gel chromatography using a gradient of ethyl acetate/dichloromethane (8% to 11% gradient) as eluent, which afforded 97 mg (100%) of 5-((2-benzyloxy-benzoylamino)methyl)-2-(tert-butoxyoxalyl-amino)-6-(4-methoxy-benzyl)-4,5,6,7-tetrahydro-thieno[2,3-c]pyridine-3-carboxylic acid tert-butyl ester.

¹H-NMR (400 MHz, CDCl₃) δ 12.54 (s, 1H), 8.41-8.39 (m, 1H), 8.23 (dd, 1H, J=8 Hz and J=2 Hz), 7.46-7.41 (m, 1H), 7.35-7.33 (m, 2H), 7.29-7.27 (m, 3H), 7.13-7.05 (m, 4H), 6.72 (d, 2H, J=8 Hz), 5.19 (s, 2H), 3.84-3.75 (m, 1H), 3.76 (s, 3H), 3.57-3.31 (m, 4H), 3.22-3.12 (m, 2H), 2.88 (dd, 1H, J=18 Hz and J=5 Hz), 2.61 (dd, 1H, J=18 Hz and J=5 Hz), 1.62 (s, 9H), 1.59 (s, 9H).

5-((2-Benzyloxy-benzoylamino)methyl)-2-(tert-butoxyoxalyl-amino)-6-(4-methoxy-benzyl)-4,5,6,7-tetrahydro-thieno[2,3-c]pyridine-3-carboxylic acid tert-butyl ester (97 mg, 0.13 mmol) was dissolved in a solution of 10% formic acid/methanol (3 ml). Palladium on activated carbon (10%, 25 mg) was added and the reaction stirred at ambient temperature for 24 hours. TLC analysis indicated that the reaction was incomplete. Additional catalyst (25 mg) was added and the reaction stirred for another 24 hours. Again, the reaction was judged incomplete by TLC and another 50 mg of catalyst was added and the reaction stirred for 72 hours.

The solution was filtered through celite and the filter cake washed with hot methanol and ethyl acetate. The filtrate was concentrated in vacuo, to the residue was added diethyl ether (4 ml) and the solution allowed to stand overnight. The precipitate was collected by filtration to give 12.5 mg (18%) of 2-(tert-butoxyoxalyl-amino)-5-((2-hydroxy-benzoylamino)methyl)-4,5,6,7-tetrahydro-thieno[2,3-c]pyridine-3-carboxylic acid tert-butyl ester.

¹H-NMR (400 MHz, CDCl₃) δ 12.47 (s, 1H), 12.18 (s, 1H), 10.26-10.03 (m, 2H), 8.67 (bs, 1H), 7.94-7.91 (m, 1H), 7.35-7.31 (m, 1H), 6.90 (d, 2H, J=8 Hz), 4.46-3.10 (m, 7H), 1.61 (s, 9H), 1.58 (s, 9H).

LC-MS: Rₜ=3.03 min, m/z: 532 [M+H]⁺.

2-(tert-Butoxyoxalyl-amino)-5-((2-hydroxy-benzoylamino)methyl)-4,5,6,7-tetrahydro-thieno[2,3-c]pyridine-3-carboxylic acid tert-butyl ester (12 mg, 0.022 mmol) was dissolved in a solution of 50% trifluoroacetic acid/dichloromethane (1 ml) and stirred at ambient temperature for 16 hours. The trifluoroacetic acid solution was added dropwise to diethyl ether and stirred for 1 hour. The precipitate was filtered off, which afforded 10 mg (85%) of the title compound as a solid.

¹H-NMR (400 MHz, DMSO) δ 12.21 (s, 1H), 9.19-9.08 (m, 2H), 7.87-7.85 (m, 1H), 7.42 (t, 1H, J=8 Hz), 6.92 (d, 2H, J=8 Hz), 4.36-2.43 (m, 7H, partially obscured by water)

LC-MS: Rₜ=1.32 min, m/z: 420 [M+H]⁺.

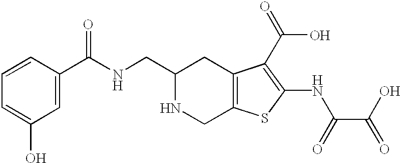

5-((3-Hydroxy-benzoylamino)methyl)-2-(oxalyl-amino)-4,5,6,7-tetrahydro-thieno[2,3-c]Pyridine-3-carboxylic Acid

A solution of 3-hydroxybenzoic acid (1.01 g, 7.3 mmol) was dissolved in anhydrous dichloromethane (20 ml) and placed under nitrogen atmosphere. Diisopropylethylamine (5.7 ml, 32.9 mmol) was added, followed by additional dichloromethane (10 ml) for complete dissolution. The solution was cooled in an ice-bath and chloromethyl methyl ether (1.38 ml, 18.3 mmol) was added. The reaction was stirred and allowed to gradually warm to ambient temperature. The reaction mixture was then stirred at ambient temperature for 48 hours, the solvents evaporated in vacuo and the residue partitioned between ethyl acetate (75 ml) and water (25 ml). The layers were separated and the organic phase washed with 1% hydrochloric acid (3×15 ml), 1N sodium hydroxide (2×10 ml), and brine (10 ml). The organic layer was dried (MgSO₄), filtered, and evaporated in vacuo affording 1.2 g (73%) of 3-methoxymethoxy-benzoic acid methoxymethyl ester as an oil.

¹H-NMR (300 MHz, CDCl₃) δ 7.73-7.70 (m, 2H), 7.36 (t, 1H, J=8 Hz), 7.26-7.22 (m, 1H), 5.46 (s, 2H), 5.21 (s, 2H), 3.53 (s, 2H), 3.46 (s, 2H)

3-Methoxymethoxy-benzoic acid methoxymethyl ester (0.647 g, 2.86 mmol) was dissolved in methanol (10 ml). Sodium hydroxide (0.17 g, 7.15 mmol) dissolved in water (1 ml) was added to the solution. The reaction was stirred for 48 hours, concentrated to dryness in vacuo. The residue was dissolved in water (15 ml) and acidified with 1N hydrochloric acid until a precipitate formed and the pH was 5. The aqueous solution was extracted with ethyl acetate (15 ml), acidified with additional 1N hydrochloric acid, and extracted again with ethyl acetate (15 ml).

The combined organic layers were washed with brine (10 ml), dried (MgSO₄), filtered, and evaporated in vacuo to give 0.48 g (93%) of 3-methoxy-methoxy-benzoic acid as a solid.

¹H-NMR (400 MHz, CDCl₃) δ 7.78-7.76 (m, 2H), 7.39 (t, 1H, J=8 Hz), 7.30-7.27 (m, 1H), 5.24 (s, 2H), 3.50 (s, 2H).

To a solution of 3-methoxy-methoxy-benzoic acid (91 mg, 0.50 mmol) and 1-(3-dimethylaminopropyl)-3-ethylcarbodiimide hydrochloride (96 mg, 0.65 mmol) in anhydrous acetonitrile (4 ml) was added triethylamine (0.16 ml, 1.5 mmol), followed by 2-amino-5-aminomethyl-6-(4-methoxy-benzyl)-4,5,6,7-tetrahydro-thieno[2,3-c]pyridine-3-carboxylic acid tert-butyl ester (155 mg, 0.384 mmol). The reaction was stirred under nitrogen atmosphere for 16 hours. The volatiles were removed in vacuo and the residue partitioned between ethyl acetate (25 ml) and water (10 ml). The layers were separated and the organic phase washed with 0.5 N hydrochloric acid (2×5 ml), saturated sodium bicarbonate (5 ml), and brine (5 ml). The organic layer was dried (MgSO₄), filtered, and concentrated in vacuo, which afforded 0.14 g of crude 2-amino-5-((3-methoxymethoxy-benzoylamino)methyl)-6-(4-methoxy-benzyl)-4,5,6,7-tetrahydro-thieno[2,3-c]pyridine-3-carboxylic acid tert-butyl ester as an oil.

¹H-NMR (300 MHz, CDCl₃) δ 7.47 (s, 1H), 7.37-7.35 (m, 2H), 7.23-7.20 (m, 3H), 6.99-6.96 (bm, 1H), 6.84 (d, 2H, J=8 Hz), 5.96 (s, 2H), 5.22 (s, 2H), 3.78 (s, 3H), 3.75-3.54 (m, 5H), 3.48 (s, 3H), 3.42-3.23 (m, 2H), 2.95-2.87 (m, 1H), 2.63-2.55 (m, 1H)

LC-MS: Rₜ=3.07 min, m/z: 568 [M+H]⁺.

To a solution of crude 2-amino-5-((3-methoxymethoxy-benzoylamino)-methyl)-6-(4-methoxy-benzyl)-4,5,6,7-tetrahydro-thieno[2,3-c]pyridine-3-carboxylic acid tert-butyl ester (141 mg, 0.248 mmol) in anhydrous dichloromethane (5 ml) was added imidazol-1-yl-oxo-acetic acid tert-butyl ester (0.15 g, 0.744 mmol). The reaction was stirred at ambient temperature for 16 hours, concentrated in vacuo and the crude material purified by silica gel chromatography using a gradient of ethyl acetate/dichloromethane (5% to 15% gradient) as eluent, which afforded 0.12 g of 2-(tert-butoxyoxalyl-amino)-5-((3-methoxymethoxy-benzoylamino)methyl)-6-(4-methoxy-benzyl)-4,5,6,7-tetrahydro-thieno[2,3-c]pyridine-3-carboxylic acid tert-butyl ester as an oil. The overall yield of the last two steps was 45%.

¹H-NMR (300 MHz, CDCl₃) δ 12.54 (s, 1H), 7.47 (s, 1H), 7.37-7.35 (m, 2H), 7.21-7.18 (m, 3H), 6.96-6.93 (bm, 1H), 6.84 (d, 2H, J=8 Hz), 5.22 (s, 2H), 3.87-3.67 (m, 4H), 3.78 (s, 3H), 3.58-3.54 (m, 1H), 3.48 (s, 3H), 3.42-3.32 (m, 2H), 3.82-2.95 (m, 1H), 2.71-2.64 (m, 1H), 1.60 (s, 18H)

LC-MS: Rₜ=1.47 min, m/z: 696 [M+H]⁺.

2-(tert-Butoxyoxalyl-amino)-5-((3-methoxymethoxy-benzoylamino)methyl)-6-(4-methoxy-benzyl)-4,5,6,7-tetrahydro-thieno[2,3-c]pyridine-3-carboxylic acid tert-butyl ester (0.12 g, 0.17 mmol) was dissolved in a solution of 10% formic acid/methanol (4 ml). Palladium on activated carbon (10%, 0.10 g) was added and the reaction stirred at ambient temperature for 72 hours. The solution was filtered through celite and the filter cake washed with hot methanol and ethyl acetate. The filtrate was evaporated in vacuo and the residue was purified by radial silica gel chromatography (methanol/dichloromethane, 1%) affording 18 mg (18%) of 2-(tert-butoxyoxalyl-amino)-5-((3-methoxymethoxy-benzoylamino)methyl)-4,5,6,7-tetrahydro-thieno[2,3-c]pyridine-3-carboxylic acid tert-butyl ester.

¹H-NMR (400 MHz, CDCl₃) δ 12.52 (s, 1H), 7.49-7.48 (m, 1H), 7.42-7.33 (m, 2H), 7.19-7.16 (m, 1H), 6.86-6.84 (m, 1H), 5.22 (s, 2H), 4.03-3.91 (m, 3H), 3.49 (s, 3H), 3.20-3.00 (m, 3H), 2.51-2.44 (m, 1H), 1.61 (s, 9H), 1.59 (s, 9H)

LC-MS: Rₜ=1.29 min, m/z: 576 [M+H]⁺.

2-(tert-Butoxyoxalyl-amino)-5-((3-methoxymethoxy-benzoylamino)methyl)-4,5,6,7-tetrahydro-thieno[2,3-c]pyridine-3-carboxylic acid tert-butyl ester (17 mg, 0.03 mmol) was dissolved in a solution of 50% trifluoroacetic acid/dichloromethane (1 ml) and stirred at ambient temperature for 16 hours. Adding the trifluoroacetic acid solution dropwise to diethyl ether precipitated the product. The suspension was stirred for 1 hour, the solid filtered off and washed with diethyl ether, which afforded 8 mg (53%) of the title compound as a solid.

¹H-NMR (400 MHz, DMSO-d₆) δ 12.31 (s, 1H), 9.69 (s, 1H), 9.19 (bs, 2H), 8.73 (s, 1H), 7.31-7.24 (m, 3H), 6.92 (d, 1H, J=8 Hz), 4.39-4.23 (m, 2H), 3.65-3.54 (m, 2H), 3.37-3.18 (m, 1H, partially obscured by water), 2.87-2.81 (m, 1H), 2.65-2.43 (m, 1H, partially obscured by DMSO)

LC-MS: Rₜ=0.52 min, m/z: 420 [M+H]⁺.

General Chiral Synthesis

4-Oxo-1-((S)-1-phenyl-ethyl)-piperidine-(R)-2-carboxylic acid ethyl ester

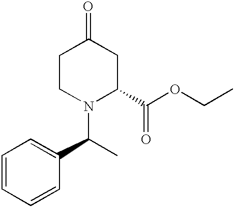

Dichloromethane (11) and mol sieves 3 Å (113 g) and amine (S)-(−)α-methyl-benzylamin (71.7 ml) were mixed in a 2 l three-necked bottle cooled to −5° C (using a ethanol/water/ice bath). A 50% solution of ethylglyoxylate in toluene (117.6 ml) was added drop wise over 20 min., keeping the temperature between −5° C. and 0° C. The mixture was stirred for 0.5 hour before it was cooled to −30° C. Trifluoroacetic acid (45.2 ml) was added over 3-4 minutes. Boron trifluoride diethyl ether (69.8 ml) was added drop wise over 5 minutes at −55° C. The ice bath was removed and the mixture was allowed to warm up to −45° C. whereupon 2-(trimethylsilyloxy)-1,3-butadiene (100 ml) was added drop wise over 10 minutes. During the addition the mixture was cooled and the temperature kept below −20° C. The above additions are all exothermic hence the cooling bath should have sufficient capacity to remove the heat generated during the rapid addition. The reaction mixture was stirred for 2 hours at −15° C. and 1 hour at 0° C. and then poured on ice/water and stirred for 15 minutes. Solid sodium hydrogen carbonate was added until pH 7-8. The mixture was stirred overnight at room temperature. The layers wee separated and the aqueous phase extracted with dichloromethane. The combined organic phases were filtered through a plug of silica eluting with dichloromethane. The relevant fractions were concentrated in vacuo. The residue was dissolved in hot heptane and cooled. This leaves a yellowish gummy material on the side of the flask and crystals starts forming. The heptane solution was heated again to dissolve crystals, leaving the gummy material on the side of the flask and the mixture was filtered hot. The heptane solution was cooled to room temperature and the precipitate was filtered off and dried in vacuo, which afforded 38 g of 4-oxo-1-((S)-1-phenyl-ethyl)piperidine-(R)-2-carboxylic acid ethyl ester as a solid.

The filtrate was put in a refrigerator and a second crop was formed which was less pure and needed recrystallization from heptane to yield another 7,5 g of 4-oxo-1-((S)-1-phenyl-ethyl)-piperidine-(R)-2-carboxylic acid ethyl ester.

4,4-Diethoxy-1-((S)-1-phenyl-ethyl)piperidine-(S)-2-carboxylic Acid Ethyl Ester

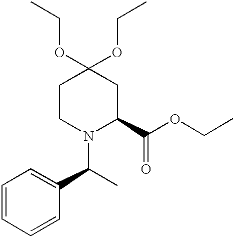

The mother liquor from the above crystallization was concentrated in vacuo. 5.0 g of the resulting material (18.16 mmol) was dissolved in ethanol (100 ml) and triethylorthoformate (26.9 g, 181.6 mmol) and para-toluen-4-sulphonic acid (6.9 g, 36.32 mmol) was added. The reaction was stirred at room temperature for 16 hours before the mixture was poured on aqueous sodium hydrogen carbonate (200 ml) and extracted with ethyl acetate (4×75 ml). The combined extracts were concentrated in vacuo and purified by column chromatography (SiO₂, Flash 40, petrol ether-ethyl acetate 10:1). Collection of the first band (R_{f}=0.68) gave 1.14 g (18%) of 4,4-diethoxy-1-((S)-1-phenyl-ethyl)-piperidine-(R)-2-carboxylic acid ethyl ester and collection of the second band (R_{f}=0.4) gave 3.60 g (57%) of the title compound.

4,4-Diethoxy-1-((S)-1-phenyl-ethyl)-piperidine-(R)-2-carboxylic acid ethyl ester

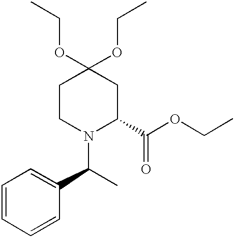

4-Oxo-1-((S)-1-phenyl-ethyl)-piperidine-(R)-2-carboxylic acid ethyl ester (11.0 g, 0.040 mmol) was dissolved in a 1:1 mixture of triethyl orthoformate and ethanol (140 ml) and para-toluene-4-sulphonic acid (15.2 g, 80 mmol) was added and the reaction mixture was stirred for 16 hours. The reaction mixture was neutralized with sodium bicarbonate (to pH 7-8), and extracted with dichloromethane (3×100 ml), dried (MgSO₄), filtered and concentrated in vacuo. The residue was purified by column chromatography (SiO₂, petrol ether/ethyl acetate 10:1), which afforded 12.0 g (86%) of the title compound as an oil.

4,4-Diethoxy-1-((S)1-phenyl-ethyl)-(R)-2-hydroxymethyl-piperidine

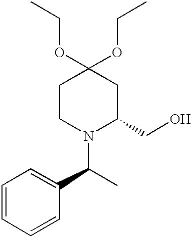

To a solution of 4,4-diethoxy-1-((S)-1-phenyl-ethyl)-piperidine-(R)-2-carboxylic acid ethyl ester (36.0 g, 0.103 mol) in dry diethyl ether (150 ml) was added a suspension of lithium aluminum hydride (5.88 g, 0.155 mol) in dry diethyl ether (300 ml) under an atmosphere of nitrogen at such a rate that the solution gently reflux. The reaction mixture was stirred over night before it was cooled to 0° C. and ethyl acetate (30 ml) was added drop wise to destroy excess lithium aluminum hydride. After stirring for another 0.5 hour, water (12 ml) was added drop wise. After stirring for 10-15 min the precipitate was filtered off through celite and the filter cage was washed with plenty of diethyl ether. The filtrate was washed with brine (100 ml), dried (MgSO₄), filtered and the solvent evaporated in vacuo, which afforded 30 g (95%) of the title compound as an oil.

4,4-Diethoxy-1-((S)-1-phenyl-ethyl)-(R)-2-phthalimidomethyl-piperidine

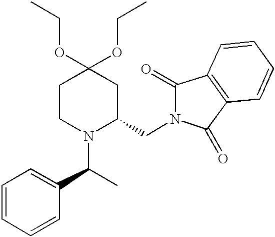

A solution of 4,4-diethoxy-1-((S)1-phenyl-ethyl)-(R)-2-hydroxymethyl-piperidine (65.35 g, 0.213 mmol), triphenylphosphine (61.3 g, 0.234 mol) and phthalimide (34.4 g, 0.234 mol) in tetrahydrofuran (700 ml) cooled to 0° C. was added diethyl azodicarboxylate over the course of 1.5 hour. The reaction mixture was stirred at 0° C. for another 2 hours before the solvent was removed in vacuo. The residue was dissolved in hot heptane-toluene (3:2) (650 ml) before it was cooled on an ice bath. The precipitate consisting of triphenyl phosphine oxide was filtered off and washed with heptane. The filtrate was concentrated in vacuo and the residue subjected to column chromatography using a mixture of toluene-ethyl acetate-heptane (3:1:3) as eluent. The solvent was evaporated in vacuo whereupon a viscous oil was obtained. Upon addition of light petrol ether the product crystallized to give 67.4 g (73%) of the title compound as a solid.

4,4-Diethoxy-1-((S)-1-phenyl-ethyl)-(S)-2-phthalimidomethyl-piperidine

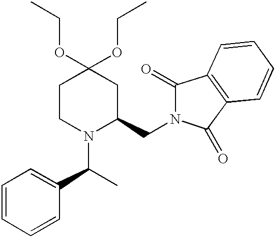

A suspension of 4,4-diethoxy-1-((S)1-phenyl-ethyl)-(S)-2-hydroxymethyl-piperidine (20 g, 65 mmol), triphenylphosphine (18.76 g, 72 mmol) and phthalimide (10.52 g, 0.072 mol) in tetrahydrofuran (200 ml) cooled to 0° C. was added diethyl azodicarboxylate over the course of 1 hour. The reaction mixture was stirred at 0° C. for another 2 hours before the solvent was removed in vacuo. The residue was dissolve in a hot mixture of heptane/toluene (3:2) (100 ml) before it was cooled on an ice bath. The precipitate was filtered off and washed with heptane. The filtrate was concentrated in vacuo and subjected to column chromatography using a mixture of toluene/ethyl acetate/heptane (3:1:3) as eluent. The solvent was evaporated in vacuo whereupon a viscous oil was obtained. Upon addition of light petrol ether (250 ml) the product crystallized to give 24 g (85%) of the title compound as a solid.

4-Oxo-1-((S)-1-phenyl-ethyl)-2-(S)-phthalimidomethyl-piperidine

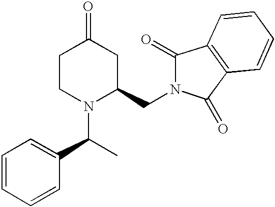

4,4-Diethoxy-1-(1-(S)-phenyl-ethyl)-2-(S)-phthalimidomethyl-piperidine (4.0 g, 9.2 mmol) was dissolved in a mixture of trifluoroacetic acid/water (9:1) (100 ml) at 0° C. and stirred for 2 hours at this temperature. The mixture was basified with half saturated aqueous sodium carbonate, extracted with ethyl acetate and dried (MgSO₄) for 2 hours and filtered. The solvent was removed in vacuo and the residue was left in a vacuum own at 40° C. for two days, which afforded 3.23 g (98%) of 4-oxo-1-((S)-1-phenyl-ethyl)-2-(S)-phthalimidomethyl-piperidine as a solid.

2-Amino-5-(S)-(1,3-dioxo-1,3-dihydro-isoindol-2-ylmethyl)-6-(1-(S)-phenyl-ethyl)-4,5,6,7-tetrahydrothieno[2,3-c]pyridine-3-carboxylic acid tert-butyl ester and

2-Amino-7-(R)-(1,3-dioxo-1,3-dihydro-isoindol-2-ylmethyl)-6-(1-(S)-phenyl-ethyl)-4,5,6,7-tetrahydrothieno[2,3-c] pyridine-3-carboxylic acid tert-butyl ester

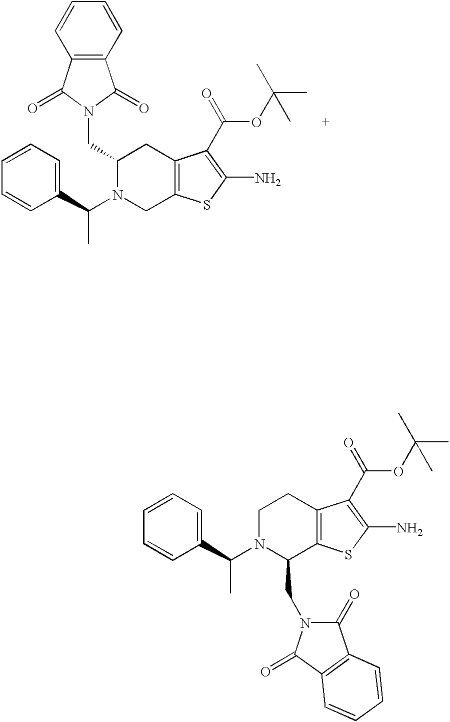

A mixture of 4-oxo-1-(1-(R)-phenyl-ethyl)-2-(S)-(phthalimidomethyl-piperidine (17.28 g, 47.73 mmol), tert-butylcyanoacetat (7.41 g, 52.17 mmol), sulphur (1.71 g, 52.17 mmol) and morpholine (8.31 g, 95.46 mmol) in ethanol (150 ml) was heated under an atmosphere of nitrogen at 50° C. The volatiles were removed in vacuo and the residue was subjected to column chromatography on silica gel using a mixture of heptane/ethyl acetate (5:1) as eluent. The band consisting of a mixture of 5- and 7-isomer was collected and purified on a reverse phase (C₁₈) column using a Flash 40 system. The residue was applied in a minimum volume of acetonitrile and eluted with a mixture of 40% acetonitrile in water containing 0.1% trifluoroacetic acid. When the first isomer (the 5-isomer) was collected the eluent was changed to 50% acetonitrile in water with 0.1% trifluoroacetic acid and the 7-isomer was collected. Yield of: 2-amino-5-(S)-(1,3-dioxo-1,3-dihydro-isoindol-2-ylmethyl)-6-(1-(S)-phenyl-ethyl)-4,5,6,7-tetrahydrothieno[2,3-c]pyridine-3-carboxylic acid tert-butyl ester was 7.96 g.

Yield of: 2-Amino-7-(R)-(1,3-dioxo-1,3-dihydro-isoindol-2-ylmethyl)-6-(1-(S)-phenyl-ethyl)-4,5,6,7-tetrahydrothieno[2,3-c]pyridine-3-carboxylic acid tert-butyl ester was 3.72 g (47% total of 5- and 7-isomer).

2-Amino-5-(S)-aminomethyl-6-(1-(S)-phenyl-ethyl)-4,5,6,7-tetrahydro-thieno[2,3-c]pyridine-3-carboxylic Acid tert-butyl Ester

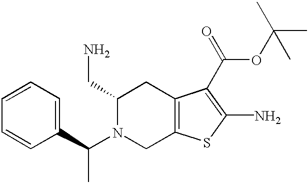

2-Amino-5-(S)-(1,3-dioxo-1,3-dihydro-isoindol-2-ylmethyl)-6-(1-(S)-phenyl-ethyl)-4,5,6,7-tetrahydrothieno[2,3-c]pyridine-3-carboxylic acid tert-butyl ester (7.96 g, 15.4 mmol) and hydrazine hydrate (3.85 g, 77.0 mmol) in ethanol (250 ml) was stirred for 16 hours at room temperature. The solvent was removed in vacuo and the white material obtained was extracted with diethyl ether (3×200 ml), the fractions were combined and the solvent was removed in vacuo to give 5.9 g (100%) of the title compound as a solid.

Method A:

General Procedure for Amide Formation:

To a solution of a relevant carboxylic acid (0.71 mmol) in N,N-dimethylformamide (5.0 ml) was added 1-(3-dimethylaminopropyl)-3-ethylcarbodiimide hydrochloride (136 mg, 0.71 mmol) and [1,2,3]triazolo[4,5-b]pyridin-3-ol (96 mg, 0.71 mmol) and the mixture was stirred for 5 minutes at room temperature before a solution of diisopropylethylamine (121 l, 0.71 mmol) and 2-amino-7-(S)-aminomethyl-6-(1-(S)-phenyl-ethyl)-4,5,6,7-tetrahydrothieno[2,3-c] pyridine-3-carboxylic acid tert-butyl ester (250 mg, 0.645 mmol) in N,N-dimethylformamide (5 ml) was added. The reaction was stirred 2 hours at room temperature before the solvent was removed in vacuo and the residue partitioned between ethyl acetate and water. The aqueous phase was extracted with ethyl acetate and the combined organic phases were dried (MgSO₄), filtered and the solvent evaporated in vacuo. The residue was purified by column chromatography using a mixture of ethyl acetate and petroleum ether as eluent, which afforded pure amide derivatives.

Method B:

General Procedure for Formation of Oxalamic Acid Tert-butyl Ester

To a solution of the fused 2-amino-thiophene in dichloromethane was added imidazol-1-yl-oxo-acetic acid tert-butyl ester (3 eq.) and triethylamine (3 eq.) and the reaction mixture was stirred 16 hours before it was transferred to a separation funnel and washed with aqueous sodium hydrogencarbonate. After drying (MgSO₄) the solvent was removed in vacuo and the crude product was purified by column chromatography using a mixture of ethyl acetate and petroleum ether as eluent.

Method C:

General Procedure for Hydrogenolysis of the N-α-methyl Benzyl Protection Group

Method Ca:

The α-methyl benzyl protected amine was dissolved in a mixture of methanol/formic acid (9:1) (app. 10 ml/100 mg α-methyl benzyl protected amine).

The solution was degassed by purging with nitrogen for 1 minute before 10% Pd/C (50% H₂O content) was added. The reaction mixture was stirred at room temperature until TLC showed that all the starting material was consumed (typical 1-4 days). The Pd/C was filtered off using Celite and the filter cage was washed with plenty of methanol. The filtrate was concentrated in vacuo and the residue partitioned between ethyl acetate and water. The organic phase was separated, dried (MgSO₄) and filtered. The solvent was removed in vacuo and the crude product was purified using column chromatography (SiO₂, and a mixture of ethyl acetate/methanol as eluent).

Method Cb:

The α-methyl benzyl protected amine was dissolved in a mixture of isopropyl alcohol/formic acid (9:1) (app. 10 ml/100 mg α-methyl benzyl protected amine). The solution was degassed by purging with nitrogen for 1 minute before 10% Pd/C (dry) was added. The reaction mixture was stirred at room temperature until TLC showed that all the starting material was consumed (typical 14 days). The Pd/C was filtered off using Celite and the filter cage was washed with plenty of methanol. The filtrate was concentrated in vacuo and partitioned between ethyl acetate and water. The organic phase was separated, dried (MgSO₄) and filtered. The solvent was removed in vacuo and the crude product was purified using column chromatography (SiO₂, and a mixture of ethyl acetate/methanol as eluent)

Method D:

General Procedure for Deprotection of Carboxylic Acid di-tert-butyl Esters

The carboxylic acid tert-butyl ester was dissolved in a mixture of trifluoroacetic acid/dichloromethane (1:1) (app. 1 ml/100 mg carboxylic acid tert-butyl ester). The reaction mixture was stirred for 16 hours at room temperature before diethyl ether (2× the reaction volume) was added drop-wise. The precipitate was filtered off/spun down on a centrifuge and washed with diethyl ether to give analytical pure title compound.

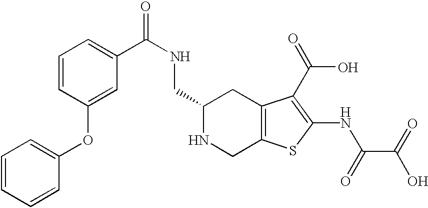

2-(Oxalyl-amino)-5-(S)-((3-phenoxy-benzoylamino)methyl)-4,5,6,7-tetrahydro-thieno[2,3-c]pyridine-3-carboxylic Acid

The title compound was prepared using 3-phenoxy-benzoic acid and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

LC-MS: m/z: 496 [M+H]⁺, Rₜ=2.00 min

Calculated for C₂₄H₂₁N₃O₇S, 1.5xC₂HF₃O₂

C, 48.65%; H, 3.40%; N, 6.30%. Found:

C, 48.26%; H, 3.42%; N, 6.09%

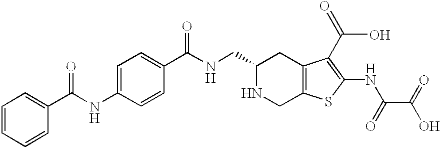

5-((4-Benzoylamino-benzoylamino)methyl)-2-(oxalyl-amino)-4,5,6,7-tetrahydro-thieno[2,3-c]pyridine-3-carboxylic Acid

The title compound was prepared using 4-benzoylamino-benzoic acid and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

Calculated for C₂₅H₂₂N₄O₇S, I xH₂O, 1xC₂HF₃O₂

C, 49.54%; H, 3.85%; N, 8.56%. Found:

C, 49.26%; H, 3.96%; N, 8.27%

### Example 22

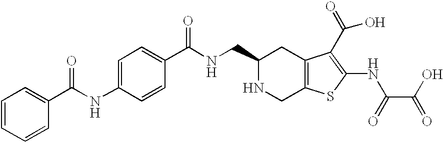

5-(R)-((4-Benzoylamino-benzoylamino)methyl)-2-(oxalyl-amino)-4,5,6,7-tetrahydro-thieno[2,3-c]pyridine-3-carboxylic Acid

The title compound was prepared using 4-benzoylamino-benzoic acid and 2-amino-5-(R)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

Calculated for C₂₅H₂₂N₄O₇S, 1xH₂O, 1xC₂HF₃O₂

C, 49.54%; H, 3.85%; N, 8.56%. Found:

C, 49.79%; H, 4.13%; N, 8.42%.

### Example 23

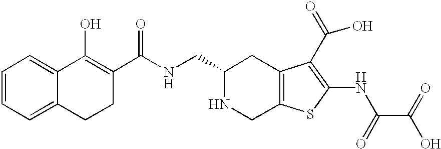

5-(S)-(((1-Hydroxy-naphthalene-2-carbonyl)amino)methyl)-2-(oxalyl-amino)-4,5,6,7-tetrahydro-thieno[2,3-c]pyridine-3-carboxylic Acid

The title compound was prepared using 1-hydroxy-naphthalene-2-carboxylic acid and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

Calculated for C₂₂H₁₉N₃O₇S, 1xH₂O, 0.6xC₂HF₃O₂

C, 50.13%; H, 3.92%; N, 7.56%. Found:

C, 50.18%; H, 4.09%; N, 7.43%

### Example 24

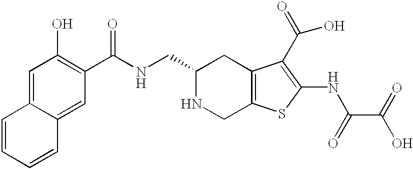

5-(S)-(((3-Hydroxy-naphthalene-2-carbonyl)amino)methyl)-2-(oxalyl-amino) 4,5,6,7-tetrahydro-thieno[2,3-c]pyridine-3-carboxylic Acid

The title compound was prepared using 3-hydroxy-naphthalene-2-carboxylic acid and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

Calculated for C₂₂H₁₉N₃O₇S, 1xH₂O, 0.6xC₂HF₃O₂

C, 50.13%; H, 3.92%; N, 7.56%. Found:

C, 50.31%; H, 4.13%; N, 7.49%

### Example 25

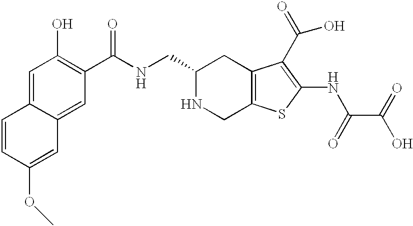

5-(S)-(((3-Hydroxy-7-methoxy-naphthalene-2-carbonyl)amino)methyl)-2-(oxalyl-amino)-4,5,6,7-tetrahydro-thieno[2,3-c]pyridine-3-carboxylic Acid

The title compound was prepared using 3-hydroxy-7-methoxy-naphthalene-2-carboxylic acid and 2-amino-5-(S)-aminomethyl-6-(1-(S)-phenyl-ethyl)4,5,6,7-tetrahydro-thieno[2,3-c]pyridine-3-carboxylic acid tert-butyl ester as the starting material and Method A, B, C and D as described above.

Calculated for C₂₂H₁₉N₃O₇S, 1xH₂O, 0.7xC₂HF₃O₂

C, 49.06%; H, 4.00%; N, 7.03%. Found:

C, 49.30%; H, 4.37%; N, 6.66%

### Example 26 (52-1257)

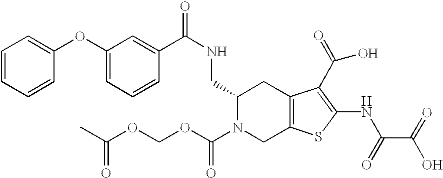

2-(Oxalyl-amino)-5-(S)-((3-phenoxy-benzoylamino)methyl)-4,5,6,7-tetrahydro-thieno[2,3-c]pyridine-3,6-dicarboxylic acid 6-acetoxymethyl Ester

The title compound was synthesised using the above 3-phenoxy-benzoylamino compound and Method A, B, C as described above and the following steps

(S)-2-(tert-Butoxyoxalyl-amino)-5-((3-phenoxy-benoylamino)methyl)-4,5,6,7-tetrahydrothieno[2,3-c]pyridine-3-carboxylic acid tert-butyl ester (100 mg, 0.165 mmol) and acetoxymethyl-p-nitrophenyl carbonate (46 mg, 0.182 mmol) was heated to 50° C. in acetonitrile (5 mL) for 6 hours. The temperature was lowered to 40° C. and the reaction mixture was kept at this temperature for 3 days under N₂. The solvent was removed in vacuo and the residue purified by flash column chromatography (SiO₂, petrol ether-ethyl acetate (1:1) affording 69 mg (58%) of (S)-2-(tert-butoxyoxalyl-amino)-5-((3-phenoxy-benzoylamino)methyl)-4,7-dihydro-5H-thieno[2,3-c]pyridine-3,6-dicarboxylic acid 6-acetoxymethyl ester 3-tert-butyl ester as an oil.

(S)-2-(tert-butoxyoxalyl-amino)-5-((3-phenoxy-benzoylamino)methyl)-4,7-dihydro-5H-thieno[2,3-c]pyridine-3,6-dicarboxylic acid 6-acetoxymethyl ester 3-tert-butyl ester (69 mg) was treated as described in Method D described above and the title compound was obtained in 69% yield.

LC-MS: m/z: 612 [M+H]⁺, Rₜ=3.80 min

### Example 27

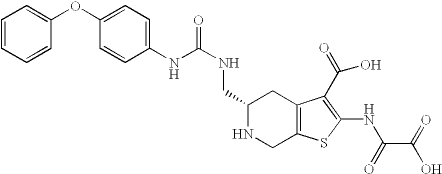

2-(Oxalyl-amino)-5-(S)-(3-(4-phenoxy-phenyl)ureidomethyl)-4,5,6,7-tetrahydro-thieno[2,3-c]pyridin-3-carboxylic Acid

To a solution of 2-amino-5-(S)-aminomethyl-6-(1-(S)-phenyl-ethyl)-4,5,6,7-tetrahydro-thieno[2,3-c]pyridine-3-carboxylic acid tert-butyl ester (500 mg, 1.29 mmol) in dichloromethane (5 ml) was added a solution of 4-phenoxyphenyl isocyanate (272 mg, 1.29 mmol) in dichloromethane (5 ml) over the course of 5 min. The reaction mixture was stirred for 45 min at room temperature before the solvent was removed in vacuo, which afforded 772 mg (100%) of 2-amino-5-(S)-(3-(4-phenoxy-phenyl)-ureidomethyl)-6-(1-(R)-phenyl-ethyl)-4,5,6,7-tetrahydro-thieno[2,3-c]pyridine-3-carboxylic acid tert-butyl ester which was used without further purification.

The title compound was obtained using the above ureido-compound and Method B, C and D as described above.

LC-MS m/z: 511 [M+H]⁺, Rₜ=2.13 min

Calculated for C₂₄H₂₂N₄O₇S, 1xH₂O and 1xC₂HF₃O₂

C, 48.60%; H, 3.92%; N, 8.72%. Found:

C, 48.95%; H, 3.80%; N, 8.50%

### Example 28

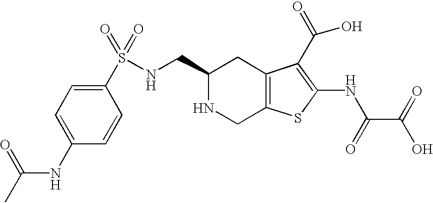

5-(R)-((4-Acetylamino-benzenesulfonylamino)methyl)-2-(oxalyl-amino)-4,5,6,7-tetrahydro-thieno[2,3-c]pyridine-3-carboxylic Acid

The title compound was prepared using 4-acetylamino-benzenesulfonyl chloride and 2-amino-5-(R)-aminomethyl-6-(1-(S)-phenyl-ethyl)-4,5,6,7-tetrahydro-thieno[2,3-c]pyridine-3-carboxylic acid tert-butyl ester as the starting material. Formation of the sulpfonamide was performed in pyridine at room temperature using a slight excess of the sulfonyl chloride followed by Method B, C and D as described above.

Calculated for C₁₉H₂₀N₄O₈S₂, 1xC₂HF₃O₂

C, 41.31%; H, 3.47%; N, 9.18%. Found:

C, 42.59%; H, 4.21%; N, 9.04%

### Example 29

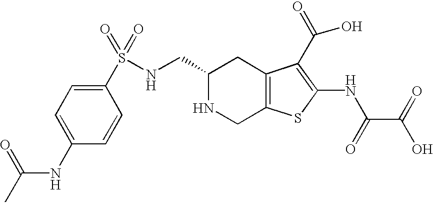

5-(S)-((4-Acetylamino-benzenesulfonylamino)methyl)-2-(oxalyl-amino)-4,5,6,7-tetrahydro-thieno[2,3-c]pyridine-3-carboxylic Acid

The title compound was prepared using 4-acetylamino-benzenesulfonyl chloride and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material. Formation of the sulfon amide was performed in pyridine at room temperature using a slight excess of the sulfonyl chloride followed by Method B, C and D as described above.

LC-MS: m/z: 497 [M+H]⁺, Rₜ=1.08 min

Calculated for C₁₉H₂₀N₄O₈S₂, 0.5xH₂O and 4/3xC₂HF₃O₂

C, 39.76%; H, 3.39%; N, 8.56%. Found:

C, 39.71%; H, 3.76%; N, 8.57%

### Example 30 (52-1267-1A)

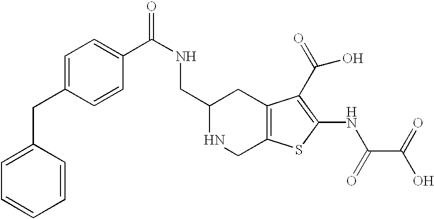

5-((4-Benzyl-benzoylamino)methyl)-2-(oxalyl-amino)-4,5,6,7-tetrahydro-thieno[2,3-c]pyridine-3-carboxylic Acid

The title compound was prepared in a similar way as described in Example 1 using 4-benzyl-benzoic acid and 2-amino-5-aminomethyl-6-(4-methoxy-benzyl)-4,5,6,7-tetrahydro-thieno[2,3-c]pyridine-3-carboxylic acid tert-butyl ester as the starting material.

¹H-NMR (300 MHz, DMSO-d₆): δ 12.31 (bs, 1H), 9.2 (bs, 2H), 8.75 (bt, 1H), 7.72 (d, 2H), 7.35-7.14 (m, 6H), 4.44-4.24 (m, 2H), 3.99 (s, 2H), 3.73-3.57 (m, 3H), 3.22 (d, 1H), 2.86 (dd, 1H).

LC-MS: m/z: 494 [M+H]⁺.

### Example 31 (52-1268-1A)

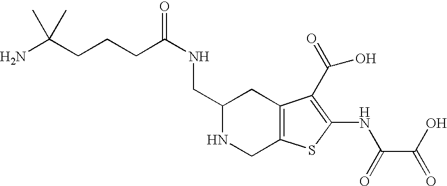

5-((5-Amino-5-methyl-hexanoylamino)methyl)-2-(oxalyl-amino)-4,5,6,7-tetrahydro-thieno[2,3-c]pyridine-3-carboxylic Acid

The title compound was prepared in a similar way as described in Example 1 using 5-tert-butoxycarbonylamino-5-methyl-hex-2-enoic acid (prepared as described by Hansen et al. in J. Med. Chem. 41; (1998); 3705-3714) and 2-amino-5-aminomethyl-6-(4-methoxy-benzyl)-4,5,6,7-tetrahydro-thieno[2,3-c]pyridine-3-carboxylic acid tert-butyl ester as the starting material.

¹H-NMR (300 MHz, DMSO-d₆): δ 12.31 (bs, 1H), 9.3 (bs, 1H), 9.15 (bs, 1H), 8.24 (bt, 1H), 7.78 (bs, 3H), 4.42-4.24 (m, 2H), 3.59-3.38 (m, 3H), 3.19 (dd, 1H), 2.79 (dd, 1H), 2.16 (bt, 2H), 1.52 (m, 4H), 1.2 (s, 6H).

LC-MS: m/z: 427 [M+H]⁺.

### Example 32 (1293-1A)

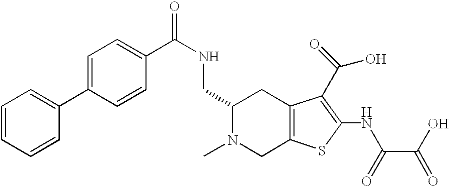

5-(S)-(((Biphenyl-4-carbonyl)amino)methyl)-6-methyl-2-(oxalyl-amino)-4,5,6,7-tetrahydro-thieno[2,3-c]pyridine-3-carboxylic acid

2-(4,4-Diethoxypiperidin-2-ylmethyl)isoindole-1,3-dione.

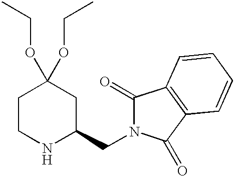

4,4-Diethoxy-1-((S)-1-phenyl-ethyl)-(S)-2-phthalimidomethyl-piperidine (15 g; prepared as in “General chiral synthesis”) was dissolved in a mixture of ethanol (150 ml) and formic acid (15 ml). Palladium on charcoal (2 g; 10%) was added under nitrogen and the mixture was stirred for 3 days. The solvent was removed in vacuo and the residue was treated with saturated aqueous sodium hydrogen-carbonate (100 ml) and methylene chloride (2×200 ml). The organic phase was dried (MgSO₄), filtered and the solvent removed in vacuo affording 12.5 g of 2-(4,4-diethoxypiperidin-2-ylmethyl)isoindole-1,3-dione.

¹H-NMR (CDCl₃, 400 Hz): δ 1.14 (m, 6H); 1.28 (m, 1H); 1.50 (dt, 1H); 1.95 (m, 1H); 2.08 (m, 1H); 2.72 (dt, 1H); 2.90 (bs, 1H); 3.05 (m, 1H); 3.17 (m, 1H); 3.39 (t, 2H); 3.51 (t, 2H); 3.71 (m, 2H); 7.68 (m, 2H); 7.82 (m, 2H).

LC-MS: m/z: 333.1 [M+H]⁺.

2-(4,4-Diethoxy-1-methylpiperidin-2-ylmethyl)isoindole-1,3-dione.

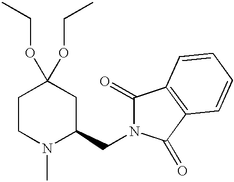

2-(4,4-diethoxypiperidin-2-ylmethyl)isoindole-1,3-dione (5.0 g 15 mmol) was dissolved in acetonitrile (50 ml) and treated with formaldehyde (1.13 ml, 15 mmol; 37% in water) and sodium triacetoxyborohydride (4.45 g, 21 mmol) under nitrogen. The mixture was stirred for 2 hours and quenched with saturated aqueous sodium hydrogen carbonate (30 ml). The solvent was partly removed in vacuo and extracted with ethyl acetate (2×100 ml) and the organic phase was dried with magnesium sulphate and the solvent was removed in vacuo leaving 4.08 g of 2-(4,4-diethoxy-1-methylpiperidin-2-ylmethyl)isoindole-1,3-dione.

¹H-NMR (CDCl₃, 400 Hz): δ 1.15 (t, 6H); 1.45 (dd, 1H); 1.68 (dt, 1H); 1.95 (m, 2H); 2.40 (dt, 1H); 2.47 (s, 3H); 2.57 (m, 1H); 2.81 (m, 1H); 3.37 (m, 2H); 3.46 (q, 2H); 3.67; 3.95 (ab-syst.; 2H).

LC-MS: m/z: 347.1 [M+H]⁺.

The title compound was prepared from 2-(4,4-diethoxy-1-methylpiperidin-2-ylmethyl)isoindole-1,3-dione using the same methods as in Example 1 and method A, B, C and D in “General chiral synthesis”.

¹H-NMR (300 MHz, DMSO-d₆): δ 12.33 (s, 1H), 10.3 (bs, 1H), 8.8 (bs, 1H), 7.97 (d, 2H), 7.81 (d, 2H), 7.74 (d, 2H), 7.50 (t, 2H), 7.42 (t, 1H), 4.75-4.36 (m, 2H), 3.97-3.51 (m, 4H), 3.28 (dd, 1H), 3.17-2.88 (m, 3H).

LC-MS: m/z: 494 [M+H]⁺.

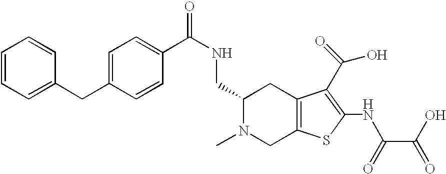

5-(S)-((4-Benzyl-benzoylamino)methyl)-6-methyl-2-(oxalyl-amino)-4,5,6,7-tetrahydro-thieno[2,3-c]pyridine-3-carboxylic Acid

The title compound was prepared from 2-(4,4-diethoxy-1-methylpiperidin-2-ylmethyl)isoindole-1,3-dione using the same methods as in Example 1 and method A, B, C and D in “General chiral synthesis”.

Calculated for C₂₆H₂₅N₃O₆S₁, 0.5xH₂O and 1xC₂HF₃O2

C, 53.3%; H, 4.3%; N, 6.6%. Found:

C, 53.5%; H, 4.5%; N, 6.6%

¹H-NMR (400 MHz, DMSO-d₆): δ 12.31 (bs, 1H), 8.72 (bt, 1H), 7.80 (d, 2H), 7.35 (d, 2H), 7.30-7.18 (m, 5H), 4.49 (bs, 2H), 4.00 (s, 2H), 3.93-3.45 (m, 4H), 3.22 (d, 1H), 3.09-2.89 (bm, 3H).

LC-MS: m/z: 508 [M+H]⁺.

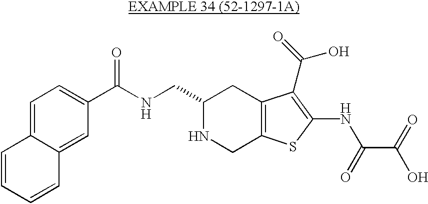

5-(S)-(((Naphthalene-2-carbonyl)amino)methyl)-2-(oxalyl-amino)-4,5,6,7-tetrahydro-thieno[2,3-c]-pyridine-3-carboxylic Acid

The title compound was prepared using naphthalene-2-carboxylic acid and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

¹H-NMR (400 MHz, DMSO-d₆): δ 13.81 (bs, 1H), 12.32 (bs, 1H), 9.26 (bs, 2H), 9.06 (bt, 1H, —CONHCH₂), 8.51 (s, 1H), 8.06-7.97 (m, 4H), 7.64-7.60 (m, 2H), 4.40 (dd, 2H), 3.79-3.66 (m, 3H), 3.52 (d, 1H), 2.95 (dd, 1H).

LC-MS: m/z: 454 [M+H]⁺.

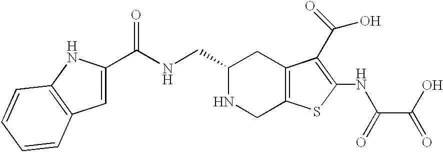

5-(S)-(((1H-Indole-2-carbonyl)amino)methyl)-2-(oxalyl-amino)-4,5,6,7-tetrahydro-thieno[2,3-c]pyridine-3-carboxylic Acid

The title compound was prepared using 1H-indole-2-carboxylic acid and 2-amino-5-(S)-aminomethyl-6-(1-(S)-phenyl-ethyl)-4,5,6,7-tetrahydro-thieno[2,3-c]pyridine-3-carboxylic acid tert-butyl ester as the starting material and Method A, B, C and D as described above.

¹H-NMR (400 MHz, DMSO-d₆): δ 13.78 (bs, 1H), 12.32 (bs, 1H), 11.63 (s, 1H), 9.29 (bs, 2H), 8.87 (bt, 1H, —CONHCH₂), 7.64 (d, 1H), 7.45 (d, 1H), 7.18 (m, 2H), 7.05 (m, 1H), 4.39 (dd, 2H), 3.71-3.63 (m, 3H), 3.26 (d, 1H), 2.90 (dd, 1H).

LC-MS: m/z: 443 [M+H]⁺.

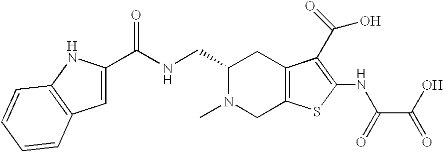

5-(S)-(((1H-Indole-2-carbonyl)amino)methyl)-6-methyl-2-(oxalyl-amino)-4,5,6,7-tetrahydro-thieno[2,3-c]pyridine-3-carboxylic Acid

The title compound was prepared from 2-(4,4-diethoxy-1-methylpiperidin-2-ylmethyl)isoindole-1,3-dione using the same methods as in Example 1 and method A, B, C and D in “General chiral synthesis”

¹H-NMR (400 MHz, DMSO-d₆): 613.78 (bs, 1H), 12.34 (bs, 1H), 11.67 (s, 1H), 10.33 (bs, 1H), 8.79 (bs, 1H, —CONHCH₂), 7.64 (d, 1H), 7.44 (d, 1H), 7.18 (m, 2H), 7.04 (m, 1H), 4.46 (dd, 2H), 3.84-3.71 (m, 3H), 3.53 (bs, 1H), 3.28 (d, 1H), 3.03 (m, 3H).

LC-MS: m/z: 457 [M+H]⁺.

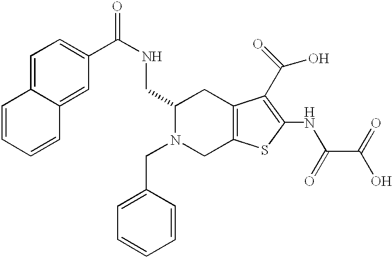

6-Benzyl-5-(S)-(((naphthalene-2-carbonyl)amino)methyl)-2-(oxalyl-amino) 4,5,6,7-tetrahydro-thieno[2,3-c]pyridine-3-carboxylic Acid

The title compound was prepared from 2-(1-benzyl-4,4-diethoxy-piperidin-2-ylmethyl)isoindole-1,3-dione using the same methods as in Example 1 and 32 and “General chiral synthesis”

¹H-NMR (400 MHz, DMSO-d₆): 13.78 (bs, 1H), 12.38 (bs, 1H), 10.45 (bs, 1H), 9.04 (bs, 1H), 8.47 (bs, 1H, —CONHCH₂), 8.00 (m, 4H), 7.63 (m, 2H), 7.48 (m, 3H), 4.31 (bs, 3H), 3.95 (bs, 3H), 3.66 (bs, 1H), 3.26 (bs, 2H).

LC-MS: m/z: 544 [M+H]⁺.

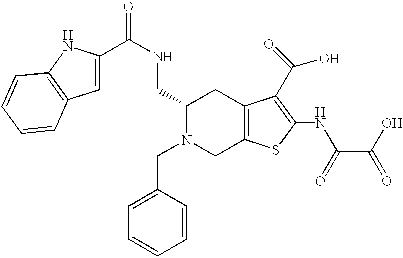

6-Benzyl-5-(S)-(((1H-indole-2-carbonyl)amino)methyl)-2-(oxalyl-amino)-4,5,6,7-tetrahydro-thieno[2,3-c]pyridine-3-carboxylic Acid

The title compound was prepared from 2-(1-benzyl-4,4-diethoxy-piperidin-2-ylmethyl)isoindole-1,3-dione using the same methods as in example 1 and 32 and “General chiral synthesis”

¹H-NMR (400 MHz, DMSO-d₆): δ 13.8 (bs, 1H), 12.38 (bs, 1H), 11.69 (s, 1H), 10.4 (bs, 1H), 8.86 (bs, 1H, —CONHCH₂), 7.65 (d, 1H), 7.48 (m, 6H), 7.20 (t, 2H), 7.16 (s, 1H), 7.05 (t, 1H), 4.7 (bm, 2H), 4.32-4.20 (m, 3H), 3.85 (m, 1H), 3.63 (m, 1H) 3.24 (bm, 2H).

LC-MS: m/z: 533 [M+H]⁺.

1. A compound of Formula 1 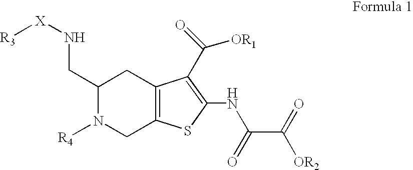
wherein
X is —C(O)— or —S(O)₂—;
R₁ and R₂ are independently hydrogen or a functional group that can be converted to hydrogen in vivo;
R₃ is hydrogen, C₁-C₆alkyl, H₂N—R₃₅—, C₂-C₆alkenyl, C₂-C₆alkynyl, aryl, aryl-R₁₀—, aryl-N(R₃₅)—, aryl-R₁₁—N(R₃₆)—, C₁-C₆alkyloxy or aryl-R₁₃—O—, wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, wherein the aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or R₃₈—R₂₃—C(O)—N(R₂₄)—;
R₄ is hydrogen, C₁-C₆alkyl, aryl-R₂₆—, R₂₇—O—C(O)—, aryl-R₂₈—O—C(O)—, R₂₉—C(O)—O—R₃₀—O—C(O)— or aryl-R₃₁—C(O)—O—R₃₂—O—C(O)—, wherein aryl is phenyl, naphthyl or thiophenyl, wherein the aryl group is optionally substituted with halogen, nitro, cyano, trihalomethyl, R₃₉—, R₃₉—R₃₃—, C₁-C₆alkyloxy or R₃₉—R₃₄—O—;
and wherein R₅, R₇, R₈, R₉, R₁₀, R₁₁, R₁₃, R₁₄, R₁₅, R₂₃, R₂₆, R₂₈, R₃₀, R₃₁, R₃₂, R₃₃, R₃₄ and R₃₅ independently are C₁-C₆alkylene, wherein R₆, R₁₂, R₁₇, R₁₈, R₂₀, R₂₂, R₂₅, R₂₇ and R₂₉ independently are C₁-C₆alkyl and wherein R₁₆, R₁₉, R₂₁, R₂₄, R₃₅ and R₃₆, and R₃₇ independently are hydrogen or C₁-C₆alkyl;
R₃₈ and R₃₉ are independently phenyl, naphthyl or thiophenyl;
or a salt thereof with a pharmaceutically acceptable acid or base, or any optical isomer or mixture of optical isomers, or any tautomeric form:

2. A compound according to claim 1, wherein R₁ and R₂ are independently hydrogen, C₁-C₆alkyl, aryl-R₅—, R₆—C(O)—O—R₇— or aryl-R₈—C(O)—O—R₉—, wherein aryl is phenyl, naphthyl or thiophenyl, and wherein the aryl group is optionally substituted with halogen, nitro, trihalomethyl, C₁-C₆alkyl or C₁-C₆alkyloxy.

3. A compound according to claim 1 wherein X is C(O).

4. A compound according to claim 1 wherein X is S(O)₂.

5. A compound according to claim 1 wherein R₁ and R₂ are independently hydrogen, C₁-C₆alkyl, aryl-R₅—, or R₆—C(O)—O—R₇—, wherein aryl is phenyl, naphthyl or thiophenyl, and wherein the aryl group is optionally substituted with halogen, nitro, trihalomethyl, C₁-C₆alkyl or C₁-C₆alkyloxy.

6. A compound according to claim 5 wherein R₁ and R₂ are independently hydrogen or C₁-C₆alkyl.

7. A compound according to claim 6 wherein R₁ and R₂ are hydrogen.

8. A compound according to claim 1 wherein R₃ is C₁-C₆alkyl, C₂-C₆alkenyl, C₂-C₆alkynyl, aryl, aryl-R₁₀—, C₁-C₆alkyloxy, H₂N—R₃₅— or aryl-R₁₃—O—, wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, and wherein the aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or R₃₈—R₂₃—C(O)—N(R₂₄)—.

9. A compound according to claim 8 wherein R₃ is C₁-C₆alkyl, C₂-C₆alkenyl, aryl, H₂N—R₃₅— or aryl-R₁₃—O— wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, and wherein the aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or
R₃₈—R₂₃—C(O)—N(R₂₄)—.

10. A compound according to claim 9 wherein R₃ is C₁-C₆alkyl or aryl, wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, and wherein the aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or R₃₈—R₂₃—C(O)—N(R₂₄)—.

11. A compound according to claim 10 wherein R₃ is aryl, wherein aryl is phenyl, biphenyl, indenyl, naphthyl, imidazolyl, 1,2,3-triazolyl, thiophenyl, pyridyl, quinolyl, isoquinolyl, indolyl or benzimidazolyl, and wherein the aryl group is optionally substituted with halogen, nitro, cyano, hydroxy, trihalomethyl, C₁-C₆alkyl, R₃₇-aryl-, R₃₈—, R₃₈—R₁₄—, C₁-C₆alkyloxy, R₃₈—O—, R₃₈—R₁₅—O—, R₃₈—N(R₁₆)—, R₁₈—C(O)—N(R₁₉)—, R₃₈—C(O)—N(R₂₁)— or R₃₈—R₂₃—C(O)—N(R₂₄)—.

12. A compound according to claim 8 wherein aryl is phenyl, biphenyl, naphthyl, 1,2,3-triazolyl, quinolyl or indolyl.

13. A compound according to claim 12 wherein aryl is phenyl, naphthyl, or indolyl.

14. A compound according to claim 13 wherein aryl is phenyl.

15. A compound according to claim 8 wherein the aryl group is substituted by halogen, hydroxy, C₁-C₆alkyl, R₃₇-aryl-, R₃₈—, R₃₈—R₁₄—, C₁-C₆alkyl-oxy, R₃₈—O—, R₃₈—R₁₅—O—, R₁₈—C(O)—N(R₁₉)—, or R₃₈—C(O)—N(R₂₁)—.

16. A compound according to claim 15 wherein the aryl group is substituted by hydroxy, C₁-C₆alkyl, R₃₈—, R₃₈—R₁₄—, C₁-C₆alkyloxy, R₃₈—O—, R₁₈—C(O)—N(R₁₉)—, or
R₃₈—C(O)—N(R₂₁)—.

17. A compound according to claim 8 wherein R₃₈ is phenyl or thiophenyl.

18. A compound according to claim 1 wherein R₄ is hydrogen, C₁-C₆alkyl, aryl-R₂₆—, R₂₉—C(O)—O—R₃₀—O—C(O)— or aryl-R₃₁—C(O)—O—R₃₂—O—C(O)—, wherein aryl is phenyl, naphthyl or thiophenyl, and wherein the aryl group is optionally substituted with halogen, nitro, cyano, trihalomethyl, R₃₉—, R₃₉—R₃₃—, C₁-C₆alkyloxy or R₃₉—R₃₄—O—.

19. A compound according to claim 18 wherein R₄ is hydrogen, C₁-C₆alkyl, or aryl-R₂₆—, wherein aryl is phenyl, naphthyl or thiophenyl, and wherein the aryl group is optionally substituted with halogen, nitro, cyano, trihalomethyl, R₃₉—, R₃₉—R₃₃—, C₁-C₆alkyloxy or R₃₉—R₃₄—O—.

20. A compound according to claim 19 wherein R₄ is hydrogen or aryl-R₂₆—, wherein aryl is phenyl, naphthyl or thiophenyl, and wherein the aryl group is optionally substituted with halogen, nitro, cyano, trihalomethyl, R₃₉—, R₃₉—R₃₃—, C₁-C₆alkyloxy or R₃₉—R₃₄—O—.

21. A compound according to claim 20 wherein R₄ is hydrogen.

22. A compound according to claim 20 wherein R₄ is aryl-R₂₆—, wherein aryl is phenyl, naphthyl or thiophenyl, and wherein the aryl group is optionally substituted with halogen, nitro, cyano, trihalomethyl, R₃₉—, R₃₉—R₃₃—, C₁-C₆alkyloxy or R₃₉—R₃₄—O—.

23. A compound according to claim 18 wherein aryl is phenyl.

24. A compound according to claim 18 wherein the aryl group is substituted by R₃₉—.

25. A compound according to claim 18 wherein the aryl group is substituted by C₁-C₆alkyloxy.

26. A compound according to claim 25 wherein aryl group is substituted by methoxy.

27. A compound according to claim 1 which is:
5-(Benzoylamino-methyl)-2-(oxalyl-amino)-4,5,6,7-tetrahydro-thieno[2,3-c] pyridine-3-carboxylic acid;
5-(((6-Bromo-2-p-tolyl-quinoline-4-carbonyl)amino)methyl)-6-(4-methoxy-benzyl)-2-(oxalyl-amino)-4,5,6,7-tetrahydro-thieno[2,3-c]pyridine-3-carboxylic acid;
5-(((5-Benzyloxy-1H-indole-2-carbonyl)amino)methyl)-6-(4-methoxy-benzyl)-2-(oxalyl-amino)-4,5,6,7-tetrahydro-thieno[2,3-c]pyridine-3-carboxylic acid;
5-((4-Benzoylamino-benzoylamino)methyl)-6-(4-methoxy-benzyl)-2-(oxalyl-amino)-4,5,6,7-tetrahydro-thieno[2,3-c]pyridine-3-carboxylic acid;
5-((4-Benzoylamino-benzoylamino)methyl)-2-(oxalyl-amino)-4,5,6,7-tetrahydro-thieno[
2,3-c]pyridine-3-carboxylic acid;
6-(4-Methoxy-benzyl)-5-(((5-methyl-2-phenyl-2H-[1,2,3]triazole-4-carbonyl)amino)methyl)-2-(oxalyl-amino)-4,5,6,7-tetrahydro-thieno[2,3-c]pyridine-3-carboxylic acid;
5-((4-Ethoxy-2-hydroxy-benzoylamino)methyl)-6-(4-methoxy-benzyl)-2-(oxalyl-amino)-4,5,6,7-tetrahydro-thieno[2,3-c]pyridine-3-carboxylic acid;
5-(((Biphenyl-4-carbonyl)amino)methyl)-6-(4-methoxy-benzyl)-2-(oxalyl-amino)-4,5,6,7-tetrahydro-thieno[2,3-c]pyridine-3-carboxylic acid;
5-(((Biphenyl-4-carbonyl)amino)methyl)-2-(oxalyl-amino)-4,5,6,7-tetrahydro-thieno[
2,3-c]pyridine-3-carboxylic acid;
5-((4-Ethoxy-2-hydroxy-benzoylamino)methyl)-2-(oxalyl-amino)-4,5,6,7-tetrahydro-thieno[2,3-c]pyridine-3-carboxylic acid;
5-(((5-Methyl-2-phenyl-2H-[1,2,3]triazole-4-carbonyl)amino)methyl)-2-(oxalyl-amino)-4,5,6,7-tetrahydro-thieno[2,3-c]pyridin-3-carboxylic acid;
5-((3-Biphenyl-4-yl-acryloylamino)methyl)-6-(4-methoxy-benzyl)-2-(oxalyl-amino)-4,5,6,7-tetrahydro-thieno[2,3-c]pyridin-3-carboxylic acid;
5-(((1H-Indole-2-carbonyl)amino)methyl)-6-(4-methoxy-benzyl)-2-(oxalyl-amino)-4,5,6,7-tetrahydro-thieno[2,3-c]pyridin-3-carboxylic acid;
5-(((1H-Indole-2-carbonyl)amino)methyl)-2-(oxalyl-amino)-4,5,6,7-tetrahydro-thieno[
2,3-c]pyridine-3-carboxylic acid;
5-((3-Biphenyl-4-yl-propionylamino)methyl)-2-(oxalyl-amino)-4,5,6,7-tetrahydro-thieno[
2,3-c]pyridine-3-carboxylic acid;
6-(4-Methoxy-benzyl)-5-(((naphthalene-1-carbonyl)amino)methyl)-2-(oxalyl-amino)-4,5,6,7-tetrahydro-thieno[2,3-c]pyridine-3-carboxylic acid;
5-(Benzyloxycarbonylamino-methyl)-2-(oxalyl-amino)-4,5,6,7-tetrahydro-thieno[
2,3-c]pyridine-3-carboxylic acid;
7-(Benzyloxycarbonylamino-methyl)-2-(oxalyl-amino)-4,5,6,7-tetrahydro-thieno[
2,3-c]pyridine-3-carboxylic acid;
5-((2-Hydroxy-benzoylamino)methyl)-2-(oxalyl-amino)-4,5,6,7-tetrahydro-thieno[
2,3-c]pyridine-3-carboxylic acid;
5-((3-Hydroxy-benzoylamino)methyl)-2-(oxalyl-amino)-4,5,6,7-tetrahydro-thieno[
2,3-c] pyridine-3-carboxylic acid;
2-(Oxalyl-amino)-5-(S)-((3-phenoxy-benzoylamino)methyl)-4,5,6,7-tetrahydro-thieno[
2,3-c]pyridine-3-carboxylic acid;
5-((4-Benzoylamino-benzoylamino)methyl)-2-(oxalyl-amino)-4,5,6,7-tetrahydro-thieno[
2,3-c]pyridine-3-carboxylic acid;
5-(R)-((4-Benzoylamino-benzoylamino)methyl)-2-(oxalyl-amino)-4,5,6,7-tetrahydro-thieno[2,3-c]pyridine-3-carboxylic acid;
5-(S)-(((1-Hydroxy-naphthalene-2-carbonyl)amino)methyl)-2-(oxalyl-amino)-4,5,6,7-tetrahydro-thieno[2,3-c]pyridine-3-carboxylic acid;
5-(S)-(((3-Hydroxy-naphthalene-2-carbonyl)amino)methyl)-2-(oxalyl-amino)-4,5,6,7-tetrahydro-thieno[2,3-c]pyridine-3-carboxylic acid;
5-(S)-(((3-Hydroxy-7-methoxy-naphthalene-2-carbonyl)amino)methyl)-2-(oxalyl-amino)-4,5,6,7-tetrahydro-thieno[2,3-c]pyridine-3-carboxylic acid;
2-(Oxalyl-amino)-5-(S)-((3-phenoxy-benzoylamino)methyl)-4,5,6,7-tetrahydro-thieno[
2,3-c]pyridine-3,6-dicarboxylic acid 6-acetoxymethyl ester;
2-(Oxalyl-amino)-5-(S)-(3-(4-phenoxy-phenyl)ureidomethyl)-4,5,6,7-tetrahydro-thieno[
2,3-c]pyridin-3-carboxylic acid;
5-((4-Benzyl-benzoylamino)methyl)-2-(oxalyl-amino)-4,5,6,7-tetrahydro-thieno[
2,3-c] pyridine-3-carboxylic acid;
5-((5-Amino-5-methyl-hexanoylamino)methyl)-2-(oxalyl-amino)-4,5,6,7-tetrahydro-thieno[2,3-c]pyridine-3-carboxylic acid;
5-(S)-(((Biphenyl-4-carbonyl)amino)methyl)-6-methyl-2-(oxalyl-amino)-4,5,6,7-tetrahydro-thieno[2,3-c]pyridine-3-carboxylic acid;
5-(S)-((4-Benzyl-benzoylamino)methyl)-6-methyl-2-(oxalyl-amino)-4,5,6,7-tetrahydro-thieno[2,3-c]pyridine-3-carboxylic acid;
5-(S)-(((Naphthalene-2-carbonyl)amino)methyl)-2-(oxalyl-amino)-4,5,6,7-tetrahydro-thieno[2,3-c]-pyridine-3-carboxylic acid;
5-(S)-(((1H-Indole-2-carbonyl)amino)methyl)-2-(oxalyl-amino)-4,5,6,7-tetrahydro-thieno[
2,3-c]pyridine-3-carboxylic acid;
5-(S)-(((1H-Indole-2-carbonyl)amino)methyl)-6-methyl-2-(oxalyl-amino)-4,5,6,7-tetrahydro-thieno[2,3-c]pyridine-3-carboxylic acid;
6-Benzyl-5-(S)-(((naphthalene-2-carbonyl)amino)methyl)-2-(oxalyl-amino)-4,5,6,7-tetrahydro-thieno[2,3-c]pyridine-3-carboxylic acid; or
6-Benzyl-5-(S)-(((1H-indole-2-carbonyl)amino)methyl)-2-(oxalyl-amino)-4,5,6,7-tetrahydro-thieno[2,3-c] pyridine-3-carboxylic acid;
or a pharmaceutically acceptable salt thereof.
